(19) United States
(12) Patent Application Publication
(10) Pub. No.: US 2025/0092331 A1
(43) Pub. Date: Mar. 20, 2025

(54) COMPOSITIONS COMPRISING 4-ISOPROPYL-1-METHYL-7-OXABICYCLO[2.2.1]HEPTAN-2-OL OR ESTERS OR ETHERS THEREOF AND THEIR USE AS AROMA CHEMICALS

(71) Applicant: BASF SE, Ludwigshafen am Rhein (DE)

(72) Inventors: Miriam BRU ROIG, Lampertheim (DE); Florian GARLICHS, Lampertheim (DE); Ralf PELZER, Lampertheim (DE); Fabian HUNDEMER, Ludwigshafen am Rhein (DE); Stefan BENSON, Ludwigshafen am Rhein (DE)

(21) Appl. No.: 18/832,528

(22) PCT Filed: Jan. 25, 2023
(86) PCT No.: PCT/EP2023/051826

§ 371 Date: Jul. 24, 2024

(30) Foreign Application Priority Data
Jan. 26, 2022 (EP) 22153455.5

(51) Int. Cl. C11B 9/00; C07D 307/00

(52) U.S. Cl. CPC C11B 9/0076; C07D 307/00; C11B 9/0034

(57) ABSTRACT

The present invention relates to the use of compositions comprising 4-isopropyl-1-methyl-7-oxabicyclo[2.2.1]heptan-2-ol or specific esters or ethers thereof and 3-isopropyl-6-methyl-cyclohex-2-en-1-one, or of 4-isopropyl-1-methyl-7-oxabic yclo[2.2.1]heptan-2-ol or specific esters or ethers thereof as aroma chemicals or for modifying and/or enhancing the aroma of a composition. The invention relates moreover to compositions comprising 4-isopropyl-1-methyl-7-oxabicyclo[2.2.1]heptan-2-ol or specific esters or ethers thereof and 3-isopropyl-6-methyl-cyclohex-2-en-1-one, to specific compositions comprising 4-isopropyl-1-methyl-7-oxabicyclo[2.2.1]heptan-2-ol, and to specific esters or ethers of 4-isopropyl-1-methyl-7-oxabicyclo[2.2.1]heptan-2-ol.

Compositions comprising 4-isopropyl-1-methyl-7-oxabicyclo[2.2.1]heptan-2-ol or esters or ethers thereof and their use as aroma chemicals

The present invention relates to the use of compositions comprising 4-isopropyl-1-methyl-7-oxabicyclo[2.2.1]heptan-2-ol or specific esters or ethers thereof and 3-isopropyl-6-methyl-cyclohex-2-en-1-one or of 4-isopropyl-1-methyl-7-oxabicyclo[2.2.1]heptan-2-ol or specific esters or ethers thereof as aroma chemicals or for modifying and/or enhancing the aroma of a composition. The invention relates moreover to compositions comprising 4-isopropyl-1-methyl-7-oxabicyclo[2.2.1]heptan-2-ol or specific esters or ethers thereof and 3-isopropyl-6-methyl-cyclohex-2-en-1-one, to specific compositions comprising 4-isopropyl-1-methyl-7-oxabicyclo[2.2.1]heptan-2-01, and to specific esters or ethers of 4-isopropyl-1-methyl-7-oxabicyclo[2.2.1]heptan-2-01.

### BACKGROUND OF THE INVENTION

Aroma chemicals, especially fragrances, are of great interest especially in the field of cosmetics and cleaning and laundry compositions. Fragrances of natural origin are mostly expensive, often limited in their available amount and, on account of fluctuations in environmental conditions, are also subject to variations in their content, purity etc. To circumvent these undesirable factors, it is therefore of great interest to create synthetic substances which have sensory properties that resemble more expensive natural fragrances or which have novel and interesting sensory profiles.

Despite a large number of already existing aroma chemicals, there is a constant need for new components in order to be able to satisfy the multitude of properties desired for extremely diverse areas of application. These include, firstly, the sensory properties, i.e. the compounds should have advantageous odiferous (olfactory) or gustatory properties. Furthermore, aroma chemicals should also have additional positive secondary properties, such as e.g. an efficient preparation method, the possibility of providing better sensory profiles as a result of synergistic effects with other aroma chemicals, a higher stability in a wide range of compositions as well as under certain application conditions, a higher extendability and/or a better staying power.

However, since even small changes in chemical structure bring about massive changes in the sensory properties such as odor and also taste, the targeted search for substances with certain sensory properties such as a certain odor is extremely difficult. The search for new fragrances and flavorings is therefore in most cases difficult and laborious without knowing whether a substance with the desired odor and/or taste will even actually be found.

Moreover, as a result of the predictable exhaustion of fossil fuels and the ecological and economical imperative to reduce waste and CO₂ emissions, it has become of ever increasing importance to maximize the efficiency of industrial production processes and at the same time to make a more reasonable use of unavoidable organic side products than just using them as a fuel or combustion material, as is presently done in most cases. Even if this fueling/combustion is used for the production of energy and thus serves at least a purpose, this use is not satisfactory, as it wastes potentially valuable materials.

3-Isopropyl-6-methyl-cyclohex-2-en-1-one, also known as carvenone, is a monocyclic monoterpene which is described to have a minty odor. 4-Isopropyl-1-methyl-7-oxabicyclo[2.2.1]heptan-2-ol is also known as 2-hydroxy-1,4-cineole.

JPH 01261327A relates to a perfume composition comprising 1-menthol and 2-hydroxy-1,8-cineole or 2-hydroxy-1,4-cineole. The 2-hydroxycineoles are used to enhance the refreshing sensation of menthol and improve its persistency. As such, they are however described as devoid of interesting characteristics in terms of aroma.

U.S. Pat. No. 4,853,089 relates to a process for purifying exo-2-hydroxy-1,4-cineole contaminated with ketones. In examples 6 and 7, mixtures containing i.a. 75.6 or 67.7 wt.-% of exo-2-hydroxy-1,4-cineole and 10.3 or 12.8 wt.-% of carvenone are subjected to said purification process.

M. Mertens et al. describe in Flavour Fragr. J. 2009, 24, 279-300 the volatile constituents of frankincense. Volatiles found in pyrolysed B. carteri are reported to contain i.a. 2-hydroxy-1,4-cineole.

S. N. Garg et al. describe in Phytochemistry 1989, 28(2), 634-636 essential oil from rhizomes of Ferula Jaeschkeana. One of the components thereof is 2β-hydroxy-1,4-cineole.

A. F. M. Barton et al. describe in J. Agric. Food Chem. 2010, 58, 10147-10155 the synthesis and herbicidal activity of various cineole derivatives. Inter alia, the conversion of 2-hydroxy-1,4-cineole into various esters and ethers thereof is described.

2-Hydroxy-1,4-cineole is the direct precursor in the synthesis of the herbicide cinmethylin. 2-Hydroxy-1,4-cineole can in turn be obtained by epoxidation of 1,4-terpinen-4-ol to the epoxide compound of the following formula:

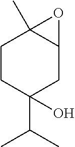

followed by acid-catalyzed rearrangement. This path is described, for example, in WO 2018/210662, EP 0081892 or EP 0081893. Isolation and purification of 2-hydroxy-1,4-cineole affords a waste stream containing as major component carvenone and minor amounts of 2-hydroxy-1,4-cineole.

It was the object of the present invention to provide new aroma chemicals. Besides, these substances should be combinable with other aroma chemicals, allowing the creation of novel advantageous sensory profiles. In addition, the process for the preparation of these new aroma chemicals should be easy and efficient to allow their fast, economic and environmentally friendly manufacturing. In particular, however, they should be available from by-products of industrial processes which otherwise would be discarded or brought to a low-value use, such as the production of energy.

These and further objects are achieved by compositions comprising the compounds of formulae (I) and (II) as shown below, by specific compositions comprising only compounds of formula (I) and also by specific compounds (I). The compositions comprising the compounds of formulae (I) and (II) are available from the production of cinmethylin from the epoxide compound shown above, which is in turn obtained from terpinen-4-ol, a compound obtainable from natural, renewable sources.

### SUMMARY OF THE INVENTION

The invention relates in one aspect to the use of a composition (also termed composition A in the following) comprising

(a) a compound of the formula (I)

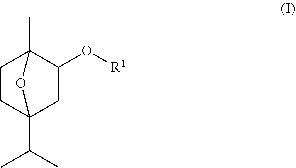

wherein
R¹ is hydrogen, C₁-C₄-alkyl, formyl or C₁-C₃-alkylcarbonyl;
or a mixture of different compounds (I), or a stereoisomer of one of these compounds (1), or a mixture of stereoisomers of one or more of these compounds (I); and
   (b) a compound of the formula (II)

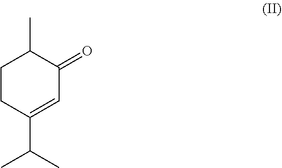

or a stereoisomer thereof or mixture of different stereoisomers thereof;
as an aroma chemical.

The invention relates also to the use of said composition A for modifying and/or enhancing the aroma of a composition; in particular for modifying and/or enhancing the fragrance impression of a composition; specifically for modifying the scent character of a fragranced ready-to-use composition.

The invention relates further to compositions A comprising a compound of the formula (I) or a mixture of different compounds (I), or a stereoisomer of one of these compounds (1), or a mixture of stereoisomers of one or more of these compounds (I); and a compound of the formula (II) or a stereoisomer thereof or mixture of different stereoisomers thereof.

The invention relates moreover to a composition (also termed composition B in the following) comprising a compound of the formula (I) or a mixture of different compounds (1) or a stereoisomer of one of these compounds (I) or a mixture of stereoisomers of one or more of these compounds (I), and at least one further component selected from the group consisting of aroma chemicals different from compounds (I) and (II), non-aroma chemical carriers, anti-oxidants and deodorant-active agents; and in particular from the group consisting of aroma chemicals different from compounds (I) and (II), surfactants, oil components, solvents, anti-oxidants and deodorant-active agents; with the proviso that R¹ is not hydrogen if the composition comprises menthol, hydroxycarvomenthone, 3,7-dimethyl-2,6-octadiene, cryptone, p-menthane-1,2-diol, dichloromethane, tetrahydrofuran, dimethylformamide, hexane or chloroform; with the proviso that R¹ is not acetyl (methylcarbonyl; —C(O)CH₃) if the composition comprises dichloromethane or hexane and with the proviso that in case that R¹ is hydrogen and the aroma chemical different from compound (I) is a compound of the formula (II) as defined in claim 1, the compound of the formula (I) and the compound of the formula (II) are present in a molar ratio of from 3:1 to 1:>1; and to the use of a compound (I) or of a mixture of different compounds (I) or of a stereoisomer of one of these compounds (1) or of a mixture of stereoisomers of one or more of these compounds (I) as an aroma chemical; or for modifying and/or enhancing the aroma of a composition; in particular for modifying and/or enhancing the fragrance impression of a composition; specifically for modifying the scent character of a fragranced ready-to-use composition, where the use does not encompass the use of the compound of the formula (I) where R¹ is hydrogen as an aroma chemical in essential oil obtained from the rhizomes of Ferula Jaeschkeana; and where the use does not encompass the use of the compound of the formula (I) where R¹ is hydrogen for modifying and/or enhancing the aroma of menthol or of essential oil obtained from the rhizomes of Ferula Jaeschkeana.

The invention relates further to a method for imparting an aroma, preferably a fragrance, to a composition, in particular to a (otherwise) ready-to-use composition, comprising incorporating a compound (I) or a mixture of different compounds (I) or a stereoisomer of one of these compounds (I) or a mixture of stereoisomers of one or more of these compounds (I); or incorporating both a compound (I) or a mixture of different compounds (I) or a stereoisomer of one of these compounds (I) or a mixture of stereoisomers of one or more of these compounds (I) and the compound (II) or a stereoisomer thereof or a mixture of different stereoisomers thereof into a composition which is to be aromatized, in particular to be fragranced.

The invention relates also to a method for modifying the scent character of an aroma chemical composition, in particular of a fragranced composition, especially of a fragranced ready-to-use composition, comprising incorporating a compound (I) or a mixture of different compounds (I) or a stereoisomer of one of these compounds (I) or a mixture of stereoisomers of one or more of these compounds (I); or incorporating both a compound (I) or a mixture of different compounds (I) or a stereoisomer of one of these compounds (I) or a mixture of stereoisomers of one or more of these compounds (1) and the compound (II) or a stereoisomer thereof or a mixture of different stereoisomers thereof into an aroma chemical composition, in particular into a fragranced composition, especially into a fragranced ready-to-use composition.

The invention further relates to a method of preparing an aroma chemical composition, in particular a fragranced composition, specifically a fragranced ready-to-use composition, comprising incorporating a compound (I) or a mixture of different compounds (I) or a stereoisomer of one of these compounds (I) or a mixture of stereoisomers of one or more of these compounds (I); or incorporating both a compound (I) or a mixture of different compounds (I) or a stereoisomer of one of these compounds (I) or a mixture of stereoisomers of one or more of these compounds (I) and the compound (II) or a stereoisomer thereof or a mixture of different stereoisomers thereof, into a composition, in particular into a ready-to-use composition.

The invention relates further to a compound (I), wherein R¹ is C₁-C₃-alkyl, formyl or ethylcarbonyl, and to the use of this compound (I) as an aroma chemical; or for modifying and/or enhancing the aroma of a composition; in particular for modifying and/or enhancing the fragrance impression of a composition; specifically for modifying the scent character of a fragranced ready-to-use composition.

### DETAILED DESCRIPTION OF THE INVENTION

### Definitions

In the context of the present invention, the term “aroma” refers to a sensory property and comprises an odor and/or a flavor.

The term “aroma chemical” denotes a substance [or in the present case a substance mixture/composition of compounds (I) and (II)] which is used to obtain an aroma impression (the term “aroma impression” is used interchangeably herein with the term “note”) and comprises its use to obtain an olfactory and/or a flavor impression. The term “olfactory impression” or “odor impression” denotes an odor impression without any positive or negative judgement, while the term “scent impression” or “fragrance impression” (used interchangeably herein) as used herein is connected to an odor impression which is generally felt as pleasant. Thus a “fragrance” or “scent” denotes an aroma chemical which predominately induces a pleasant odor impression. A flavor denotes an aroma chemical which induces a taste impression.

The term “aroma profile” denotes the overall aroma impression of an aroma chemical and is composed of the individual aroma impressions of an aroma chemical.

The term “aroma composition”, as used herein, refers to a composition which induces an aroma. The term aroma composition comprises “odor composition” and/or “flavor composition”. An odor composition is a composition which predominately induces an odor impression, whereas a flavor composition is a composition which predominantly induces a taste impression.

The term “odor composition” comprises “fragrance composition” or “scent composition” (used interchangeably herein), which predominately induce an odor impression which is generally felt as pleasant.

“Pleasant odor”, “pleasant odor impression”, “pleasant odiferous properties”, “odor impression felt as pleasant” and similar terms are hedonistic expressions which describe the niceness and conciseness of an odor impression conveyed by an aroma chemical. The more general hedonistic expressions “advantageous sensory properties” or “advantageous organoleptic properties” describe the niceness and conciseness of an organoleptic impression conveyed by an aroma chemical. In terms of the present invention, the terms “organoleptic” and “sensory” relate to olfactory or flavor properties. “Niceness” and “conciseness” are terms which are familiar to the person skilled in the art, a perfumer. Niceness generally refers to a spontaneously brought about, positively perceived, pleasant sensory impression. However, “nice” does not have to be synonymous with “sweet”. “Nice” can also be the odor of musk or sandalwood. “Conciseness” generally refers to a spontaneously brought about sensory impression which—for the same test panel—brings about a reproducibly identical reminder of something specific. For example, a substance can have an odor which is spontaneously reminiscent of that of an “apple”: the odor would then be concisely of “apples”. If this apple odor were very pleasant because the odor is reminiscent, for example, of a sweet, fully ripe apple, the odor would be termed “nice”. However, the odor of a typical apple tart can also be concise. If both reactions arise upon smelling the substance, in the example thus a nice and concise apple odor, then this substance has particularly advantageous sensory properties.

The term “odor-intensive substances” refers to substances or aroma chemicals exhibiting intense odor impressions. Intense odor impressions are to be understood as meaning those properties of aroma chemicals which permit a striking perception even in very low gas space concentrations.

The intensity can be determined via a threshold value determination. A threshold value is the concentration of a substance in the relevant gas space at which an odor impression can just still be perceived by a representative test panel, although it no longer has to be defined. A substance class which probably belongs to the most odor-intensive known substance classes, i.e. has very low odor threshold values, are thiols, whose threshold value is often in the ppb/m³ range.

The term “tenacity” describes the evaporation behavior over time of an aroma chemical. The tenacity can for example be determined by applying the aroma chemical to a test strip, and by subsequent olfactory evaluation of the odor impression of the test strip. For aroma chemicals with high tenacity the time span after which the panel can still identify an aroma impression is long.

The term “substantivity” describes the interaction of an aroma chemical with a surface, such as for example the skin or a textile, especially after subsequent treatment of the surface, such as for example washing. The substantivity can for example be determined by washing a textile with a textile detergent composition comprising the aroma chemical and subsequent olfactory evaluation of the textile directly after washing (wet textile) as well as evaluation of the dry textile after prolonged storage.

The term “stability” describes the behavior of an aroma chemical upon contact with oxygen, light and/or other substances. An aroma chemical with high stability maintains its aroma profile over a long period in time, preferably in a large variety of compositions and under various storage conditions.

In order to impart a long-lasting aroma impression to a composition or to a surface treated with a composition, the tenacity, the substantivity as well as the stability of the aroma chemical in the compositions should preferably be high.

The term “booster”, “boosting” or “boost” is used herein to describe the effect of enhancing and/or modifying the aroma of an aroma chemical or of a composition. The term “enhancing” comprises an improvement of the niceness and/or conciseness of an aroma and/or an improvement of the intensity. The term “modifying” comprises the change of an aroma profile.

Booster effects are particularly desired in fragrance composition when top-note-characterized applications are required, in which the odor is to be conveyed particularly quickly and intensively, for example in deodorants, air fresheners or in the taste sector in chewing gums.

The terms “compound” and “substance” are used synonymously throughout the invention.

The composition A comprising compounds (I) and (II) as defined above as components (a) and (b) can be a mixture consisting essentially of only the compounds (I) and (II), or can be a composition which contains further ingredients, such as those defined below, e.g. solvents or other carriers, aroma chemicals different from (I) and (II), surfactants etc. “Essentially” means that the mixture can contain minor amounts of impurities, “minor” meaning at most 5% by weight, preferably at most 2% by weight, more preferably at most 1% by weight, relative to the total weight of said mixture. In case of the use of composition A according to the invention as an aroma chemical as well as the method of using said composition A, composition A is preferably a mixture consisting essentially of only the compounds (I) and (II), or consisting essentially of only the compounds (I) and (II) and a carrier, e.g. a solvent. The carrier in this case has generally no or at most weak aroma properties which do not alter the aroma properties of the mixture of compounds (I) and (II). More preferably, in case of the use of composition A according to the invention as an aroma chemical as well as the method of using said composition A, composition A is preferably a mixture consisting essentially of only the compounds (I) and (II).

C₁-C₃-Alkyl refers to methyl, ethyl, n-propyl or isopropyl. C₁-C₄-Alky refers to methyl, ethyl, n-propyl, isopropyl, n-butyl, sec-butyl, isobutyl or tert-butyl.

Formyl is a group —C(═O)H.

C₁-C₃-Alkylcarbonyl is a group —C(═O)—R, where R is C₁-C₃-alkyl. Examples are methylcarbonyl (acetyl; —C(═O)—CH₃), ethylcarbonyl (propanoyl; —C(═O)—CH₂CH₃), n-propyl-carbonyl (n-butanoyl; —C(═O)—CH₂CH₂CH₃) and isopropylcarbonyl (isobutanoyl; —C(═O)—CH(CH₂)₃).

The term “stereoisomers” as used in context with the present invention relates specifically to geometrical isomers or optical isomers, such as enantiomers or diastereomers, the latter existing due to more than one stereogenic center in the molecule, to a (bridged) ring system or to a C═C double bond.

In terms of the present invention, the stereoisomers of the compounds (I) are also characterized as “exo” or “endo” isomers. Exo isomers are compounds (I) in which the —OR¹ group points into the same direction as the shortest bridge of the bicyclic ring system; here the oxygen ring member of the bicyclic ring, relative to the “plane” formed by the six carbon ring atoms. In the endo isomers, the —OR¹ group points into the opposite direction as the shortest bridge of the bicyclic ring system (i.e. the oxygen ring member of the bicyclic ring).

In terms of the present invention, the term “pure enantiomer” is understood as the pure enantiomer in the proper sense, i.e. where the antipode is below the detection threshold, and is also understood as a non-racemic mixture of a specific compound, where the specific enantiomer is present in an enantiomeric excess of at least 90% ee, preferably of at least 95% ee.

In terms of the present invention, the term “pure diastereomer” is understood as the pure diastereomer in the proper sense, i.e. where the other diastereomer(s) is/are below the detection threshold, and is also understood as a mixture of the diastereomers of a specific compound, where the specific diastereomer is present in an amount of at least 90%, preferably of at least 95%, based on the total amount of the diastereomers of said compound.

In the present context, the term “compound I”, “compound (I)”, “compound of formula (I)” or simply (I), when not defined as a specific stereoisomer or a specific mixture of stereoisomers, refers to the form of the compound as it is obtained in a non-stereo-selective method used for its production. The term is however also used if it is not necessary or not possible to specify in more detail the stereochemistry of the compound (1).

Analogously, the term “compound II”, “compound (II)”, “compound of formula (II)” or simply (II), when not defined as a specific stereoisomer or a specific mixture of stereoisomers, refers to the form of the compound as it is obtained in a non-stereoselective method used for its production. The term is however also used if it is not necessary or not possible to specify in more detail the stereochemistry of the compound (II).

If in the following the compound of formula (I) is defined to be a specific, defined compound (in contrast to a mixture of different compounds 1), this means that the compound contains less than 2% by weight, preferably less than 1% by weight of other compounds (I), relative to the overall weight of the specific, defined compound (I) and the optionally present other compound(s) (I).

In mixtures containing different compounds (I), these differ in the definition of the radical R¹. The mixtures can contain 2-hydroxy-1,4-cineole (i.e. compound (I) wherein R¹ is H) and one or more ethers thereof (i.e. compound (I) wherein R¹ is C₁-C₄-alkyl) or one or more esters thereof (i.e. compound (I) wherein R¹ is formyl or C₁-C₃-alkylcarbonyl) or both one or more ethers and one or more esters thereof; or the mixtures can contain two or more different ethers; or can contain two or more different esters; or can contain one or more ethers and one or more esters. Due to the preparation method in which compounds (I) in which R¹ is not hydrogen are prepared from compounds (I) in which R¹ is hydrogen or from other alcohol precursors which have to be converted into ethers or esters, such compounds may also contain compounds (I) in which R¹ is H if the reaction is not complete and/or if no isolation/purification step is carried out or not to a sufficient extent. In such mixtures, compounds (I) in which R¹ is H are generally contained in only minor amounts, such as at most 10% by weight, preferably at most 5% by weight, in particular at most 2% by weight, based on the total weight of all compounds (I) present in the mixture.

In case that compound (I) is not specified to be specific compound (I), but can also be a mixture of different compounds (I), the mixtures of stereoisomers of the compound (I) can be mixtures of different stereoisomers of a specific compound (I) or mixtures of stereoisomers of different compounds (I).

The remarks made below concerning preferred definitions of the compounds of formula (I) and (II), where applicable, as well as concerning other preferred properties of the compositions, uses and methods of the invention are valid on their own as well as preferably in combination with each other.

### EMBODIMENTS (E.X) OF THE INVENTION

General and preferred embodiments E.x are summarized in the following, non-exhaustive list. Further preferred embodiments become apparent from the paragraphs following this list.

E.1. The use of a composition (composition A) comprising
(a) a compound of the formula (I)

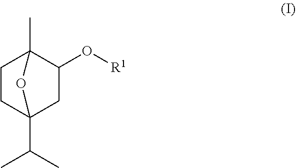

wherein
R¹ is hydrogen, C₁-C₄-alkyl, formyl or C₁-C₃-alkylcarbonyl;
or a stereoisomer thereof or a mixture of different stereoisomers thereof or
a mixture of different compounds (I); and
(b) a compound of the formula (II)

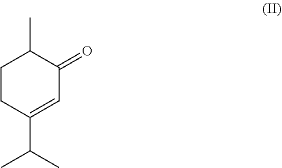

or a stereoisomer thereof or mixture of different stereoisomers thereof;
as an aroma chemical.

E.2. The use according to embodiment E.1, where in the compound of the formula (I) R¹ is formyl or C₁-C₃-alkylcarbonyl.

E.3. The use according to embodiment E.2, where in the compound of the formula (I) R¹ is C₁-C₃-alkylcarbonyl.

E.4. The use according to embodiment E.3, where in the compound of the formula (I) R¹ is methylcarbonyl or ethylcarbonyl.

E.5. The use according to embodiment E.4, where in the compound of the formula (I) R¹ is methylcarbonyl.

E.6. The use according to embodiment E.1, where in the compound of the formula (I) R¹ is C₁-C₄-alkyl.

E.7. The use according to embodiment E.6, where in the compound of the formula (I) R¹ is C₁-C₃-alkyl.

E.8. The use according to embodiment E.7, where in the compound of the formula (I) R¹ is methyl or ethyl.

E.9. The use according to embodiment E.8, where in the compound of the formula (I) R¹ is ethyl.

E.10. The use according to embodiment E.1, where in the compound of the formula (I) R¹ is hydrogen.

E.11. The use according to any of the preceding embodiments, where in the compound of the formula (I) the group —OR¹ is bound in exo-position.

E.12. The use according to any of the preceding embodiments, where the compound of the formula (I) and the compound of the formula (II) are present in a molar ratio of from 100:1 to 1:100.

E.13. The use according to embodiment E.12, where the compound of the formula (I) and the compound of the formula (II) are present in a molar ratio of from 50:1 to 1:50.

E.14. The use according to embodiment E.13, where the compound of the formula (I) and the compound of the formula (II) are present in a molar ratio of from 1:1 to 1:50.

E.15. The use according to embodiment E.14, where the compound of the formula (I) and the compound of the formula (II) are present in a molar ratio of from 1:2 to 1:40.

E.16. The use according to embodiment E.15, where the compound of the formula (I) and the compound of the formula (II) are present in a molar ratio of from 1:2 to 1:30.

E.17. The use according to embodiment E.16, where the compound of the formula (I) and the compound of the formula (II) are present in a molar ratio of from 1:5 to 1:30.

E.18. The use according to embodiment E.16, where the compound of the formula (I) and the compound of the formula (II) are present in a molar ratio of from 1:2 to 1:15 or from 1:5 to 1:15.

E.19. The use according to any of the preceding embodiments, where the composition is a mixture consisting essentially of the compounds (I) and (II) as defined in any of embodiments E.1 to E.18.

E.20. The use according to any of the preceding embodiments, as a fragrance.

E.21. The use of a composition as defined in any of embodiments E.1 to E.19 (and specifically of a mixture consisting essentially of the compounds (I) and (II) as defined in any of embodiments E.1 to E.19) for modifying and/or enhancing the aroma of a composition.

E.22. The use according to embodiment E.21, for modifying and/or enhancing the fragrance impression of a composition.

E.23. The use according to embodiment E.22, for modifying the scent character of a fragranced ready-to-use composition.

E.24. A composition (composition A) as defined in any of embodiments E.1 to E.19; i.e. comprising
(a) a compound of the formula (I) as defined in any of embodiments E.1 to E.11 or a stereoisomer thereof or a mixture of different stereoisomers thereof or a mixture of different compounds (I), and
(b) a compound of the formula (II) as defined in embodiment E.1 or a stereoisomer thereof or mixture of different stereoisomers thereof.

E.25. The composition according to embodiment E.24, where the compound of the formula (I) and the compound of the formula (II) are present in a molar ratio of from 100:1 to 1:100, preferably from 50:1 to 1:50, more preferably from 1:1 to 1:50, in particular from 1:2 to 1:30, more particularly from 1:2 to 1:15 or from 1:5 to 1:30 or from 1:5 to 1:15.

E.26. The composition according to any of embodiments E.24 or E.25, where in case that in the compound of the formula (I) R¹ is hydrogen, the compound of the formula (I) and the compound of the formula (II) are present in a molar ratio of from 3:1 to 1:100.

E.27. The composition according to embodiment E.26, where in case that in the compound of the formula (I) R¹ is hydrogen, the compound of the formula (I) and the compound of the formula (II) are present in a molar ratio of from 1:1 to 1:100.

E.28. The composition according to embodiment E.27, where in case that in the compound of the formula (I) R¹ is hydrogen, the compound of the formula (I) and the compound of the formula (II) are present in a molar ratio of from 1:1 to 1:50.

E.29. The composition according to embodiment E.28, where in case that in the compound of the formula (I) R¹ is hydrogen, the compound of the formula (I) and the compound of the formula (II) are present in a molar ratio of from 1:2 to 1:30.

E.30. The composition according to embodiment E.29, where in case that in the compound of the formula (I) R¹ is hydrogen, the compound of the formula (I) and the compound of the formula (II) are present in a molar ratio of from 1:2 to 1:15 or from 1:5 to 1:30 or from 1:5 to 1:15.

E.31. The composition according to any of embodiments E.24 to E.30, where in case that in the compound of the formula (I) R¹ is not hydrogen, the compound of the formula (I) and the compound of the formula (II) are present in a molar ratio of from 100:1 to 1:100.

E.32. The composition according to embodiment E.31, where in case that in the compound of the formula (I) R¹ is not hydrogen, the compound of the formula (I) and the compound of the formula (II) are present in a molar ratio of from 50:1 to 1:50.

E.33. The composition according to embodiment E.32, where in case that in the compound of the formula (I) R¹ is not hydrogen, the compound of the formula (I) and the compound of the formula (II) are present in a molar ratio of from 1:1 to 1:50.

E.34. The composition according to embodiment E.33, where in case that in the compound of the formula (I) R¹ is not hydrogen, the compound of the formula (I) and the compound of the formula (II) are present in a molar ratio of from 1:2 to 1:30.

E.35. The composition according to embodiment E.34, where in case that in the compound of the formula (I) R¹ is not hydrogen, the compound of the formula (I) and the compound of the formula (II) are present in a molar ratio of from 1:2 to 1:15 or from 1:5 to 1:30 or from 1:5 to 1:15.

E.36. The composition according to any of the embodiments E.24 to E.35, further comprising at least one further component selected from the group consisting of aroma chemicals different from compounds (I) and (II), non-aroma chemical carriers, anti-oxidants and deodorant-active agents.

E.37. The composition according to embodiment E.36, further comprising at least one further component selected from the group consisting of aroma chemicals different from compounds (I) and (II), surfactants, oil components, solvents, antioxidants and deodorant-active agents.

E.38. A composition (composition B) comprising a compound of the formula (I) as defined in any of embodiments E.1 to E.11, or a stereoisomer thereof or a mixture of different stereoisomers thereof or a mixture of different compounds (I), and at least one further component selected from the group consisting of aroma chemicals different from compound (I) and different from compounds (II) as defined in embodiment E.1; non-aroma chemical carriers, anti-oxidants and deodorant-active agents;
with the proviso that R¹ is not hydrogen if the composition comprises menthol, 4-hydroxycarvomenthone, 3,7-dimethyl-2,6-octadiene, cryptone, p-menthane-1,2-diol, toluene, dichloromethane, tetrahydrofuran, dimethylformamide, hexane or chloroform;
with the proviso that R¹ is not acetyl (methylcarbonyl; —C(O)CH₃) if the composition comprises dichloromethane or hexane; and
with the proviso that in case that R¹ is hydrogen and the aroma chemicals different from compound (I) is a compound of the formula (II) as defined in claim 1, the compound of the formula (I) and the compound of the formula (II) are present in a molar ratio of from 3:1 to 1:>1, preferably from 3:1 to 1:100, more preferably 1:1 to 1:100, even more preferably from 1:1 to 1:50, in particular from 1:2 to 1:30, more particularly from 1:2 to 1:15.

E.39. The composition according to embodiment E.38, where the at least one further component is selected from the group consisting of aroma chemicals different from compound (I) and (II), surfactants, oil components, solvents, anti-oxidants and deodorant-active agents.

E.40. The composition according to any of embodiments E.38 and E.39, where in the compound of the formula (I) R¹ is C₁-C₄-alkyl, formyl or C₁-C₃-alkylcarbonyl.

E.41. The composition according to embodiment E.40, where in the compound of the formula (I) R¹ is C₁-C₄-alkyl or C₁-C₃-alkylcarbonyl, preferably C₁-C₃-alkyl or acetyl.

E.42. The composition according to embodiment E.41, where in the compound of the formula (I) R¹ is C₁-C₃-alkyl.

E.43. The composition according to any of embodiments E.36 to E.42, where in case that the non-aroma chemical carriers comprise one or more solvents, these are selected from the group consisting of ethanol, isopropanol, dipropylene glycol (DPG), propylene glycol, 1,2-butylene glycol, glycerol, diethylene glycol monoethyl ether, diethyl phthalate (DEP), isopropyl myristate (IPM), triethyl citrate (TEC), benzyl benzoate and mixtures thereof.

E.44. The composition according to any of embodiments E.36 to E.43, where the aroma chemicals different from compounds (I) and (II) are selected from the group consisting of geranyl acetate, alpha-hexylcinnamaldehyde, 2-phenoxyethyl isobutyrate, dihydromyrcenol, methyl dihydrojasmonate (preferably with a content of cis isomer of more than 60 wt. %), 4,6,6,7,8,8-hexamethyl-1,3,4,6,7,8-hexahydrocyclopenta[g]benzopyran, tetrahydrolinalool, ethyllinalool, benzyl salicylate, 2-methyl-3-(4-tert-butylphenyl)propanal, cinnamyl alcohol, 4,7-methano-3a,4,5,6,7,7a-hexahydro-5-indenyl acetate, 4,7-methano-3a,4,5,6,7,7a-hexahydro-6-indenyl acetate, citronellol, citronellyl acetate, tetrahydrogeraniol, vanillin, linalyl acetate, styrolyl acetate, octahydro-2,3,8,8-tetramethyl-2-acetonaphthone, 2-acetyl-1,2,3,4,6,7,8-octahydro-2,3,8,8-tetramethylnaphthalene, hexyl salicylate, 4-tert-butylcyclohexyl acetate, 2-tert-butylcyclohexyl acetate, alpha-ionone, n-alpha-methylionone, alpha-isomethylionone, coumarin, terpinyl acetate, 2-phenylethyl alcohol, 4-(4-hydroxy-4-methylpentyl)-3-cyclohexenecarboxaldehyde, alpha-amylcinnamaldehyde, ethylene brassylate, (E)-3-methylcyclopentadec-5-enone, (Z)-3-methylcyclopentadec-5-enone, 15-pentadec-11-enolide, 15-pentadec-12-enolide, 15-cyclopentadecanolide, 1-(5,6,7,8-tetrahydro-3,5,5,6,8,8-hexamethyl-2-naphthalenyl)ethanone, 2-isobutyl-4-methyltetrahydro-2H-pyran-4-ol, 2-ethyl-4-(2,2,3-trimethyl-3-cyclopenten-1-yl)-2-buten-1-ol, cis-3-hexenyl acetate, trans-3-hexenyl acetate, trans-2/cis-6-nonadienol, 2,4-dimethyl-3-cyclohexenecarboxaldehyde, 2,4,4,7-tetramethyloct-6-en-3-one, 2,6-dimethyl-5-hepten-1-al, borneol, 3-(3-isopropylphenyl)butanal, 2-methyl-3-(3,4-methylenedioxyphenyl)propanal, 3-(4-ethylphenyl)-2,2-dimethylpropanal, 7-methyl-2H-1,5-benzodioxepin-3(4H)-one, 3,3,5-trimethylcyclohexyl acetate (preferably with a content of cis isomers of 70 wt. % or more), 2,5,5-trimethyl-1,2,3,4,4a,5,6,7-octahydronaphthalen-2-ol, 3-(4-tert-butylphenyl)-propanal, ethyl 2-methylpentanoate, ethoxymethoxycyclododecane, 2,4-dimethyl-4,4a,5,9b-tetrahydroindeno[1,2-d][1,3]dioxine, (2-tertbutylcyclohexyl) acetate, 3-[5,5,6-trimethylbicyclo[2.2.1]hept-2-yl]cyclohexan-1-ol, menthone, isomenthone, carvone, camphor, beta-ionone, beta-n-methylionone, beta-isomethylionone, alpha-irone, alpha-damascone, beta-damascone, beta-damascenone, delta-damascone, acetylated cedar wood oil, and mixtures thereof.

E.45. The composition according to any of embodiments E.24 to E.44, which is selected from the group consisting of perfume compositions, body care compositions, products for oral or dental hygiene, hygiene articles, cleaning compositions, textile detergent compositions, compositions for scent dispensers, foods, food supplements, pharmaceutical compositions and crop protection compositions.

E.46. The use of a compound of the formula (I) as defined in any of embodiments E.1 to E.11 or of a stereoisomer thereof or of a mixture of different stereoisomers thereof or of a mixture of different compounds (I), as an aroma chemical; where preferably the use does not encompass the use of the compound of the formula (I) where R¹ is hydrogen as an aroma chemical in essential oil obtained from the rhizomes of Ferula Jaeschkeana.

E.47. The use according to embodiment E.46, as a fragrance.

E.48. The use of a compound of the formula (I) as defined in any of embodiments E.1 to E.11 or of a stereoisomer thereof or of a mixture of different stereoisomers thereof or of a mixture of different compounds (I), for modifying and/or enhancing the aroma of a composition, where the use does not encompass the use of the compound of the formula (I) where R¹ is hydrogen for modifying the aroma of menthol or of essential oil obtained from the rhizomes of Ferula Jaeschkeana.

E.49. The use according to embodiment E.48, for modifying and/or enhancing the fragrance impression of a composition.

E.50. The use according to embodiment E.49, for modifying the scent character of a fragranced ready-to-use composition.

E.51. The use according to any of embodiments E.46 to E.50, where R¹ is C₁-C₄-alkyl, formyl or C₁-C₃-alkylcarbonyl.

E.52. The use according to embodiment E.51, where R¹ is C₁-C₃-alkyl.

E.53. A compound of the formula (I)

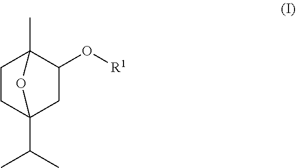

wherein
R¹ is C₁-C₃-alkyl, formyl or ethylcarbonyl;
or a stereoisomer thereof or a mixture of different stereoisomers thereof or a mixture of different compounds (I).

E.54. The compound according to embodiment E.53, where R¹ is C₁-C₃-alkyl or ethylcarbonyl.

E.55. The compound according to embodiment E.54, where R¹ is methyl, ethyl or ethylcarbonyl.

E.56. The compound according to embodiment E.55, where R¹ is ethyl.

The companies from which the above-listed aroma chemical compounds identified by tradenames and indices can be obtained are listed below.

### Composition A and Use Thereof

In a preferred embodiment, in the compound of the formula (I) R¹ is formyl or C₁-C₃-alkylcarbonyl, more preferably C₁-C₃-alkylcarbonyl, in particular methylcarbonyl (acetyl) or ethylcarbonyl (propionyl), and is specifically methylcarbonyl.

In another preferred embodiment, in the compound of the formula (I) R¹ is C₁-C₄-alkyl, more preferably C₁-C₃-alkyl, in particular methyl or ethyl, and is specifically ethyl.

In another preferred embodiment, in the compound of the formula (I) R¹ is hydrogen.

In the compound of the formula (I) the group —OR¹ can be bound in exo- or in endo-position.

Exo-position means in the present case that the bond between carbon ring atom and oxygen atom of the OR¹ group points into the same direction as the oxygen ring member of the bicyclic ring, relative to the plane formed by the six carbon ring atoms.

Endo-position means in the present case that the bond between carbon ring atom and oxygen atom of the OR¹ group points into the opposite direction as the oxygen ring member of the bicyclic ring, relative to the plane formed by the six carbon ring atoms.

In a preferred embodiment, in the compound of the formula (I) the group —OR¹ is bound in exo-position.

Preferably, the compound of the formula (I) and the compound of the formula (II) are present in a molar ratio of from 100:1 to 1:100, more preferably from 50:1 to 1:50, even more preferably from 1:1 to 1:50, particularly preferably from 1:2 to 1:40, in particular from 1:2 to 1:30, e.g. from 1:2 to 1:15 or from 1:5 to 1:30 or, especially, from 1:5 to 1:15.

However, where the composition A as such is concerned (not its use according to the invention or the method of using it), the above molar ratios apply preferably only for the case that in compounds (I) R¹ is not hydrogen. In case that R¹ is hydrogen, the compound of the formula (I) and the compound of the formula (II) are preferably present in a molar ratio of from 3:1 to 1:100, more preferably 1:1 to 1:100, even more preferably from 1:1 to 1:50, in particular from 1:2 to 1:30, more particularly from 1:2 to 1:15 or from 1:5 to 1:30 or, especially, from 1:5 to 1:15.

In an embodiment of the present invention, the composition comprising compounds (I) and (II) is a mixture consisting essentially of compounds (I) and (II). As explained above, “essentially” means that the mixture can contain minor amounts of impurities, “minor” meaning at most 5% by weight, preferably at most 2% by weight, more preferably at most 1% by weight, relative to the total weight of said mixture.

In another embodiment, composition A comprising compounds (I) and (II) comprises further constituents different from compounds (I) and (II). Preferably, in this case, the composition further comprises at least one further component selected from the group consisting of aroma chemicals different from compounds (I) and (II), non-aroma chemical carriers, anti-oxidants and deodorant-active agents; and in particular from the group consisting of aroma chemicals different from compounds (I) and (II), surfactants, oil components, solvents, anti-oxidants and deodorant-active agents. Suitable and preferred aroma chemicals different from compounds (I) and (II), non-aroma chemical carriers, surfactants, oil components, solvents, anti-oxidants and deodorant-active agents are defined below.

The compound (I) wherein R¹ is hydrogen and the compound (II) are known substances and are either commercially available or can be prepared by standard proceedings.

Preferably, the compound (I) wherein R¹ is hydrogen is obtained from a renewable source, to be more precise from terpinen-4-ol (3), which is epoxidised at the double bond to the epoxide compound (I). Acid-catalyzed rearrangement yields the compound (I) wherein R¹ is hydrogen (termed (I′) in the below scheme):

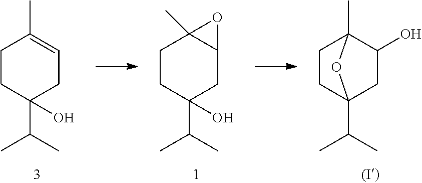

This synthetic path is described in WO 2018/210662, EP 0081892 or EP 0081893 and the literature cited therein. Terpinen-4-ol (3) can be isolated from natural sources and is available as a racemate or a non-racemic mixture in which one of the two enantiomers prevails. Since under the reaction conditions described in WO 2018/210662, EP 0081892 or EP 0081893 the two steps proceed stereoselectively, the steric information of 3 is present in (I′), too. Alternatively or additionally, the compounds 3, 1 or (I′) can be subjected to enantiomeric resolution to obtain high enantiomeric purities. Suitable methods are known in the art, e.g. the method described in WO 2018/210662 involving a selective, enzyme-catalysed acylation of just one of the enantiomers and separation from the non-acylated antipode.

Compounds (I) wherein R¹ is C₁-C₄-alkyl, formyl or C₁-C₃-alkylcarbonyl can be prepared by standard proceedings, e.g. by etherification (to obtain compounds wherein R¹ is C₁-C₄-alkyl) or esterification (to obtain compounds wherein R¹ is formyl or C₁-C₃-alkylcarbonyl) of the compound (I) wherein R¹ is hydrogen.

Ester compounds (I) wherein R¹ is formyl or C₁-C₃-alkylcarbonyl can be prepared by esterification of the compound (I) wherein R¹ is hydrogen with an acid R—C(O)OH, where R is H or C₁-C₃-alkyl, i.e. with formic acid, acetic acid, propionic acid, butyric acid or isobutyric acid, or with more active derivatives of said acids, such as the acid halides (e.g. the chloride or bromide), anhydrides or active esters thereof, under typical esterification conditions, if desired under removal of the reaction water formed (if the acid as such is used) or of the acid formed (if an anhydride is used) to enhance the reaction rate, or under neutralization (if an acid halide is used).

The esterification can be carried out in the presence of an esterification catalyst; this is especially indicated if the carboxylic acid as such is used. Suitable esterification catalysts are well known in the art and are for example metal based catalysts, e.g. iron, cadmium, cobalt, lead, zinc, antimony, magnesium, titanium and tin catalysts in the form of metals, metal oxides or metal salts, such as metal alkoxylates; or acids, e.g. mineral acids, such as sulfuric acid, hydrochloric acid or phosphoric acid; organic sulfonic acids, such as methane sulfonic acid or para-toluene sulfonic acid or strongly acidic cation exchange resins.

The term “strongly acidic cationic exchanger” refers to a cationic exchanger in the H⁺form which has strongly acidic groups. The strongly acidic groups are generally sulfonic acid groups; they are generally bonded to a polymer matrix, which can be e.g. gel-like and/or macroporous. Preference is given to styrene (co)polymers containing sulfonic acid groups, specifically to styrene-divinyl benzene copolymers containing sulfonic acid groups. Commercial examples for such cationic exchangers are Lewatit® (Lanxess), Purolite® (The Purolite Company), Dowex® (Dow Chemical Company), Amberlite® (Rohm and Haas Company), Amberlyst® (Rohm and Haas Company). Preferred strongly acidic cation exchangers are: Lewatit® K 1221, Lewatit® K 1461, Lewatit® K 2431, Lewatit® K 2620, Lewatit® K 2621, Lewatit® K 2629, Lewatit® K 2649, Amberlite® FPC 22, Amberlite® FPC 23, Amberlite® IR 120, Amberlyst® 131, Amberlyst® 15, Amberlyst® 31, Amberlyst® 35, Amberlyst® 36, Amberlyst® 39, Amberlyst® 46, Amberlyst® 70, Purolite® SGC650, Purolite® C100H, Purolite® C150H, Dowex® 50X8, Serdolit® red and Nafion® NR-50. Alternatively, the cation exchanger can be a perfluorinated ion exchange resin, sold e.g. under the Nafion® brand of DuPont.

The esterification can be carried out in the presence of a base; this is especially indicated if a carboxylic acid halide is used. Suitable bases are for example organic bases, such as tertiary amines, e.g. trimethylamine, triethylamine, tripropylamine, ethyldiisopropylamine and the like, or basic N-heterocycles, such as morpholine, pyridine, lutidine, DMAP, DABCO, DBU or DBN.

The reaction can be carried out in a solvent or neat. Suitable solvents are e.g. alkanes, such as hexane or heptane, halogenated C₁-C₄-alkanes, such as dichloromethane, chloroform or dichloroethane, cycloalkanes, such as cyclohexane, aromatic hydrocarbons, such as toluene, the xylenes, chlorobenzebne or dichlorobenzene, aliphatic ethers, such as diisopropylether or methyl-tert-butyl ether, cyclic ethers, such as tetrahydrofuran or the dioxanes, and the like. The specified solvents can be used on their own or in the form of mixtures with one another.

After completion of the reaction, the esterification product can be isolated by usual means, if necessary after neutralization (especially if an acid halide or anhydride was used as starting material or if the acid was used in excess) and/or removal of the catalyst (especially if this is solid, e.g. one of the above-mentioned metal based catalysts or ionic exchange resins), for example by distillative, extractive or chromatographic methods. If desired, the isolated product can subsequently be further purified.

Ether compounds (I) wherein R¹ is C₁-C₄-alkyl can be prepared by etherification alkylation) of the compound (I) wherein R¹ is hydrogen with an alkylation reagent R¹—X, wherein R¹ is C₁-C₄-alkyl and X represents a leaving group, such as a halogen atom, e.g. Cl, Br or I, or a sulfonate group, e.g. tosylate, mesylate, triflate or nonaflate, typically in the presence of a base.

Suitable bases can be selected from inorganic bases and organic bases. Suitable inorganic bases are for example alkali metal carbonates, e.g. Li₂CO₃, Na₂CO₃, K₂CO₃ or Cs₂CO₃, alkali metal hydroxides, e.g. LiOH, NaOH or KOH, and hydride donors, e.g. NaH, LiAIH₄ or NaBH₄. Examples for suitable organic bases correspond to those listed above, i.e. tertiary amines, e.g. trimethylamine, triethylamine, tripropylamine, ethyldiisopropylamine and the like, or basic N-heterocycles, such as morpholine, pyridine, lutidine, DMAP, DABCO, DBU or DBN.

The alkylation reaction is performed under conventional alkylation reaction conditions that are well known to the skilled person.

The reaction can be carried out in a solvent or neat. Examples for suitable solvents are listed above in context with the esterification reaction.

After completion of the reaction, the etherification product can be isolated by usual means, if necessary after neutralization (especially if X is a halogen atom or if acidic conditions were applied), for example by distillative, extractive or chromatographic methods. If desired, the isolated product can subsequently be further purified.

Compound (II) (carvenone) is commercially available.

The composition comprising compounds (I) and (II) can simply be obtained by mixing compounds (I) and (II) in the desired mixing ratio. Alternatively, a mixture of the compound (I) wherein R¹ is hydrogen and the compound (II) is used as the starting material in the esterification or etherification reaction, since compound (II) does not interfere negatively in these conversions.

The invention relates moreover to the use of the above-defined composition A comprising compounds (I) and (II), especially in form of a mixture consisting essentially of compounds (I) and (II), as an aroma chemical; in particular as a fragrance.

Specifically, a composition containing carvenone (II) and 2-hydroxy-1,4-cineole (compound (I) wherein R¹═H) in a weight ratio of ca. 87:12 is used for imparting one of the following olfactory notes: caraway, terpenic, spicy, woody.

Specifically, a composition containing carvenone (II) and 2-hydroxy-1,4-cineole (compound (I) wherein R¹═H) in a weight ratio of 99:1 to 1:99 is used for imparting one of the above olfactory notes, where with increasing amount of 2-hydroxy-1,4-cineole the scent becomes milder. Between 25:75 and 1:99, the impression is less spicy and more fruity.

Specifically, a composition containing carvenone (II) and the acetate of 2-hydroxy-1,4-cineole (compound (I) wherein R¹=—C(O)CH₃) in a weight ratio of ca. 87:12 is used for imparting one of the following olfactory notes: powdery, caraway, woody.

Specifically, a composition containing carvenone (II) and the acetate of 2-hydroxy-1,4-cineole (compound (I) wherein R¹=—C(O)CH₃) in a weight ratio of 99:1 to 1:99 is used for imparting one of the above olfactory notes, where with increasing amount of (4-isopropyl-1-methyl-7-oxabicyclo[2.2.1]heptan-2-yl) acetate the scent becomes milder. Between 25:75 and 1:99, the impression is less spicy and more fruity.

Specifically, a composition containing carvenone (II) and the ethyl ether of 2-hydroxy-1,4-cineole (compound (I) wherein R¹=CH₂CH₃)) in a weight ratio of ca. 87:12 is used for imparting one of the following olfactory notes: powdery, caraway with citrus and fruity aspects.

Specifically, a composition containing carvenone (II) and the ethyl ether of 2-hydroxy-1,4-cineole (compound (I) wherein R¹=CH₂CH₃)) in a weight ratio of 99:1 to 1:99 is used for imparting one of the above olfactory notes, where with increasing amount of 2-ethoxy-4-isopropyl-1-methyl-7-oxabicyclo[2.2.1]heptane the scent becomes milder. Between 50:50 and 1:99, the impression is less spicy and more citrus and fruity.

In addition to the olfactory properties, the compounds (I) and (II) exhibit advantageous secondary properties.

For example, they can provide better sensory profiles as a result of synergistic effects with other fragrances, which means that they can provide a booster effect for other fragrances (they give “volume” to the compositions; act as “lifters”). They are therefore suitable as boosters for other fragrances.

The invention relates also to the use of the above-defined composition A comprising compounds (I) and (II), especially in form of a mixture consisting essentially of compounds (I) and (II), for modifying and/or enhancing the aroma of a composition; in particular for modifying and/or enhancing the fragrance impression of a composition; specifically for modifying the scent character of a fragranced ready-to-use composition; and specifically to the use as a booster for other fragrances.

Booster effect means that the substances enhance and intensify in perfumery formulations the overall impression of the mixture. In the mint range, for example, it is known that menthyl methyl ether intensifies the perfumery or taste mixtures of peppermint oils and particularly in top notes brings about a considerably more intensive and more complex perception although the ether itself, being a pure substance, develops no particular intensive odor at all. In fragrance applications, Hedione® (methyl dihydrojasmonate), which as a pure substance only exhibits a light floral jasmin-note, reinforces diffusion, freshness and volume of a perfume composition as an odor booster. Booster effects are particularly desired when top-note-characterized applications are required, in which the odor impression is to be conveyed particularly quickly and intensively, for example in deodorants, air fresheners or in the taste sector in chewing gums.

To achieve such a booster effect, the compounds (I) and (II) are generally used in an overall amount of 0.1 to 20% by weight, preferably in an amount of 0.5 to 5% by weight, in particular in an amount of from 0.6 to 3% by weight, based on the total weight of the fragrance mixture.

Furthermore, compounds (I) and (II) can have further positive effects on the composition in which they are used. For example, they can enhance the overall performance of the composition into which they are incorporated, such as the stability, e.g. the formulation stability, the extendability or the staying power of the composition.

A further embodiment of the invention is directed to a method of preparing an aroma chemical composition, in particular a fragranced composition, especially a fragranced ready-to-use composition, comprising incorporating compounds (I) and (II) into the target composition, e.g. a ready-to-use composition, resulting in an aroma chemical composition, in particular in a fragranced composition, especially in a fragranced ready-to-use composition. Alternatively, the invention is directed to a method of preparing an aroma chemical composition, in particular a fragranced composition, especially a fragranced ready-to-use composition, comprising mixing the compounds (I) and (II) with at least one aroma chemical different from compounds (I) and (II) and/or with at least one non-aroma chemical carrier and/or with at least one antioxidant and/or with at least one deodorant-active agent. Suitable and preferred aroma chemicals different from compounds (I) and (II), non-aroma chemical carriers, antioxidants and deodorant-active agents are described below.

For example, the method can be carried out by mixing the compounds (I) and (II), as defined above, and at least one further component selected from the group consisting of aroma chemicals different from compounds (I) and (II), non-aroma chemical carriers, antioxidants and deodorant-active agents.

The invention is also directed to a method for modifying the scent character of an aroma chemical composition, in particular of a fragranced composition, especially of a fragranced ready-to-use composition, comprising incorporating the compounds (I) and (II) into an aroma chemical composition, in particular into a fragranced composition, especially into a fragranced ready-to-use composition.

The invention is further directed to a method for imparting an aroma, preferably a fragrance, to a composition, in particular to a (otherwise) ready-to-use composition, comprising incorporating the compounds (I) and (II) into a composition which is to be aromatized, in particular to be fragranced.

In a particular embodiment the invention is directed to a method for imparting one of the following olfactory notes: caraway, terpenic, spicy, woody; or any combination thereof to a perfume composition, body care composition, product for oral and dental hygiene, hygiene article, cleaning composition, textile detergent composition, composition for scent dispensers, food, food supplement, pharmaceutical composition or crop protection composition, which comprises including a composition containing carvenone (II) and 2-hydroxy-1,4-cineole (compound (I) wherein R¹=H) in a weight ratio of ca. 87:12 in a perfume composition, body care composition, product for oral and dental hygiene, hygiene article, cleaning composition, textile detergent composition, composition for scent dispensers, food, food supplement, pharmaceutical composition or crop protection composition.

In another particular embodiment the invention is directed to a method for imparting one of the above olfactory notes or any combination thereof to a perfume composition, body care composition, product for oral and dental hygiene, hygiene article, cleaning composition, textile detergent composition, composition for scent dispensers, food, food supplement, pharmaceutical composition or crop protection composition, which comprises including a composition containing carvenone (II) and 2-hydroxy-1,4-cineole (compound (I) wherein R¹═H) in a weight ratio of 99:1 to 1:99 in a perfume composition, body care composition, product for oral and dental hygiene, hygiene article, cleaning composition, textile detergent composition, composition for scent dispensers, food, food supplement, pharmaceutical composition or crop protection composition. With increasing amount of 2-hydroxy-1,4-cineole the scent becomes milder. Between 25:75 and 1:99, the impression is less spicy and more fruity.

In another particular embodiment the invention is directed to a method for imparting one of the following olfactory notes: powdery, caraway, woody; or any combination thereof to a perfume composition, body care composition, product for oral and dental hygiene, hygiene article, cleaning composition, textile detergent composition, composition for scent dispensers, food, food supplement, pharmaceutical composition or crop protection composition, which comprises including a composition containing carvenone (II) and the acetate of 2-hydroxy-1,4-cineole (compound (I) wherein R¹=—C(O)CH₃) in a weight ratio of ca. 87:12 in a perfume composition, body care composition, product for oral and dental hygiene, hygiene article, cleaning composition, textile detergent composition, composition for scent dispensers, food, food supplement, pharmaceutical composition or crop protection composition.

In another particular embodiment the invention is directed to a method for imparting one of the above olfactory notes or any combination thereof to a perfume composition, body care composition, product for oral and dental hygiene, hygiene article, cleaning composition, textile detergent composition, composition for scent dispensers, food, food supplement, pharmaceutical composition or crop protection composition, which comprises including a composition containing carvenone (II) and the acetate of 2-hydroxy-1,4-cineole (compound (I) wherein R¹=—C(O)CH₃) in a weight ratio of 99:1 to 1:99 in a perfume composition, body care composition, product for oral and dental hygiene, hygiene article, cleaning composition, textile detergent composition, composition for scent dispensers, food, food supplement, pharmaceutical composition or crop protection composition. With increasing amount of (4-isopropyl-1-methyl-7-oxabicyclo[2.2.1]heptan-2-yl) acetate the scent becomes milder. Between 25:75 and 1:99, the impression is less spicy and more fruity.

In another particular embodiment the invention is directed to a method for imparting one of the following olfactory notes: powdery, caraway with citrus and fruity apects; or any combination thereof to a perfume composition, body care composition, product for oral and dental hygiene, hygiene article, cleaning composition, textile detergent composition, composition for scent dispensers, food, food supplement, pharmaceutical composition or crop protection composition, which comprises including a composition containing carvenone (II) and the ethyl ether of 2-hydroxy-1,4-cineole (compound (I) wherein R¹=CH₂CH₃) in a weight ratio of ca. 87:12 in a perfume composition, body care composition, product for oral and dental hygiene, hygiene article, cleaning composition, textile detergent composition, composition for scent dispensers, food, food supplement, pharmaceutical composition or crop protection composition.

In another particular embodiment the invention is directed to a method for imparting one of the above olfactory notes or any combination thereof to a perfume composition, body care composition, product for oral and dental hygiene, hygiene article, cleaning composition, textile detergent composition, composition for scent dispensers, food, food supplement, pharmaceutical composition or crop protection composition, which comprises including a composition containing carvenone (II) and the ethyl ether of 2-hydroxy-1,4-cineole (compound (I) wherein R¹=CH₂CH₃) in a weight ratio of ca. 87:12 in a perfume composition, body care composition, product for oral and dental hygiene, hygiene article, cleaning composition, textile detergent composition, composition for scent dispensers, food, food supplement, pharmaceutical composition or crop protection composition. With increasing amount of 2-ethoxy-4-isopropyl-1-methyl-7-oxabicyclo[2.2.1]heptane the scent becomes milder. Between 50:50 and 1:99, the impression is less spicy and more citrus and fruity.

### Composition B

The invention relates moreover to a composition B comprising a compound of the formula (I) as defined above, or a stereoisomer thereof or a mixture of different stereoisomers thereof or a mixture of different compounds (I), and at least one further component selected from the group consisting of aroma chemicals different from compound (I) and (II), non-aroma chemical carriers, anti-oxidants and deodorant-active agents; and in particular from the group consisting of aroma chemicals different from compound (I) and (II), surfactants, oil components, solvents, anti-oxidants and deodorant-active agents;
preferably with the proviso that R¹ is not hydrogen if the composition comprises menthol, 4-hydroxycarvomenthone, 3,7-dimethyl-2,6-octadiene, cryptone, p-menthane-1,2-diol, toluene, dichloromethane, tetrahydrofuran, dimethylformamide, hexane or chloroform; with the proviso that R¹ is not acetyl (methylcarbonyl; —C(O)CH₃) if the composition comprises dichloromethane or hexane; and with the proviso that in case that R¹ is hydrogen and the aroma chemicals different from compound (I) is a compound of the formula (II) as defined in claim 1, the compound of the formula (I) and the compound of the formula (II) are present in a molar ratio of from 3:1 to 1:>1, preferably from 3:1 to 1:100, more preferably 1:1 to 1:100, even more preferably from 1:1 to 1:50, in particular from 1:2 to 1:30, more particularly from 1:2 to 1:15.

Composition B differs from composition A in that the presence of compound (II) is not mandatory, whereas the presence of at least one of the above-specified further components is.

In composition B, in the compound of the formula (I) R¹ is preferably C₁-C₄-alkyl, formyl or C₁-C₃-alkylcarbonyl, more preferably C₁-C₄-alkyl or C₁-C₃-alkylcarbonyl, even more preferably C₁-C₃-alkyl, in particular ethyl or methyl and specifically ethyl.

As already said in context with composition A, in a preferred embodiment, in the compound of the formula (I) the group —OR¹ is bound in exo-position.

The compound (I) can be prepared as described above in context with composition A.

Suitable aroma chemicals different from compound (I) and (II), non-aroma chemical carriers, surfactants, oil components, solvents, anti-oxidants and deodorant-active agents are listed below.

Preferably, in case that the non-aroma chemical carriers in composition B comprise one or more solvents, these are selected from the group consisting of ethanol, isopropanol, dipropylene glycol (DPG), propylene glycol, 1,2-butylene glycol, glycerol, diethylene glycol monoethyl ether, diethyl phthalate (DEP), isopropyl myristate (IPM), triethyl citrate (TEC), benzyl benzoate and mixtures thereof.

The invention relates moreover to the use of a compound of the formula (I) as defined above or of a stereoisomer thereof or of a mixture of different stereoisomers thereof or of a mixture of different compounds (I), as an aroma chemical, in particular as a fragrance; where, preferably, the use does not encompass the use of the compound of the formula (I) where R¹ is hydrogen as an aroma chemical in essential oil obtained from the rhizomes of Ferula Jaeschkeana. In this context, R¹ is preferably C₁-C₄-alkyl, formyl or C₁-C₃-alkylcarbonyl, more preferably C₁-C₄-alkyl or C₁-C₃-alkylcarbonyl, even more preferably C₁-C₃-alkyl, in particular methyl or ethyl, specifically ethyl.

Specifically, the acetate of 2-hydroxy-1,4-cineole (compound (I) wherein R¹=—C(O)CH₃) is used for imparting one of the following olfactory notes: ambrinol, animalic, civet, soft.

Specifically, the ethyl ether of 2-hydroxy-1,4-cineole (compound (I) wherein R¹═CH₂CH₃) is used for imparting one of the following olfactory notes: herbal, incense.

In addition to the olfactory properties, the compounds (I) exhibit advantageous secondary properties.

For example, they can provide better sensory profiles as a result of synergistic effects with other fragrances, which means that they can provide a booster effect for other fragrances (they give “volume” to the compositions; act as “lifters”). They are therefore suitable as boosters for other fragrances.

The invention relates also to the use of a compound (I) as defined above or of a stereoisomer thereof or of a mixture of different stereoisomers thereof or of a mixture of different compounds (I), for modifying and/or enhancing the aroma of a composition; in particular for modifying and/or enhancing the fragrance impression of a composition; specifically for modifying the scent character of a fragranced ready-to-use composition; and specifically to the use as a booster for other fragrances. Preferably, the use does not encompass the use of the compound of the formula (I) where R¹ is hydrogen for modifying and/or enhancing the aroma of menthol or for modifying and/or enhancing the aroma of essential oil obtained from the rhizomes of Ferula Jaeschkeana. In this context, R¹ is preferably C₁-C₄-alkyl, formyl or C₁-C₃-alkylcarbonyl, more preferably C₁-C₄-alkyl or C₁-C₃-alkylcarbonyl, even more preferably C₁-C₃-alkyl, in particular methyl or ethyl, specifically ethyl.

Booster effect means that the substances enhance and intensify in perfumery formulations the overall impression of the mixture. In the mint range, for example, it is known that menthyl methyl ether intensifies the perfumery or taste mixtures of peppermint oils and particularly in top notes brings about a considerably more intensive and more complex perception although the ether itself, being a pure substance, develops no particular intensive odor at all. In fragrance applications, Hedione® (methyl dihydrojasmonate), which as a pure substance only exhibits a light floral jasmin-note, reinforces diffusion, freshness and volume of a perfume composition as an odor booster. Booster effects are particularly desired when top-note-characterized applications are required, in which the odor impression is to be conveyed particularly quickly and intensively, for example in deodorants, air fresheners or in the taste sector in chewing gums.

To achieve such a booster effect, the compounds (I) are generally used in an overall amount of 0.1 to 20% by weight, preferably in an amount of 0.5 to 5% by weight, in particular in an amount of from 0.6 to 3% by weight, based on the total weight of the fragrance mixture.

Furthermore, compounds (I) can have further positive effects on the composition in which they are used. For example, they can enhance the overall performance of the composition into which they are incorporated, such as the stability, e.g. the formulation stability, the extendability or the staying power of the composition.

A further embodiment of the invention is directed to a method of preparing an aroma chemical composition, in particular a fragranced composition, especially a fragranced ready-to-use composition, comprising incorporating a compound (I) or a stereoisomer thereof or a mixture of different stereoisomers thereof or a mixture of different compounds (I) into the target composition, e.g. a ready-to-use composition, resulting in an aroma chemical composition, in particular in a fragranced composition, especially in a fragranced ready-to-use composition. Alternatively, the invention is directed to a method of preparing an aroma chemical composition, in particular a fragranced composition, especially a fragranced ready-to-use composition, comprising mixing the compound (I) or a stereoisomer thereof or a mixture of different stereoisomers thereof or a mixture of different compounds (I) with at least one aroma chemical different from compounds (I) and (II) and/or with at least one non-aroma chemical carrier and/or with at least one antioxidant and/or with at least one deodorant-active agent. Suitable and preferred aroma chemicals different from compounds (I) and (II), non-aroma chemical carriers, antioxidants and deodorant-active agents are described below.

For example, the method can be carried out by mixing the compound (I) or a stereoisomer thereof or a mixture of different stereoisomers thereof or a mixture of different compounds (I), as defined above, and at least one further component selected from the group consisting of aroma chemicals different from compounds (I) and (II), non-aroma chemical carriers, antioxidants and deodorant-active agents.

The invention is also directed to a method for modifying the scent character of an aroma chemical composition, in particular of a fragranced composition, especially of a fragranced ready-to-use composition, comprising incorporating the compound (I) or a stereoisomer thereof or a mixture of different stereoisomers thereof or a mixture of different compounds (I) into an aroma chemical composition, in particular into a fragranced composition, especially into a fragranced ready-to-use composition.

The invention is further directed to a method for imparting an aroma, preferably a fragrance, to a composition, in particular to a (otherwise) ready-to-use composition, comprising incorporating a compound (I) or a mixture of different compounds (I) or a stereoisomer of one of these compounds (I) or a mixture of stereoisomers of one or more of these compounds (I) into a composition which is to be aromatized, in particular to be fragranced.

In a particular embodiment the invention is directed to a method for imparting one of the following olfactory notes: ambrinol, animalic, civet, soft; or any combination thereof to a perfume composition, body care composition, product for oral and dental hygiene, hygiene article, cleaning composition, textile detergent composition, composition for scent dispensers, food, food supplement, pharmaceutical composition or crop protection composition, which comprises including the acetate of 2-hydroxy-1,4-cineole (compound (I) wherein R¹=—C(O)CH₃) in a perfume composition, body care composition, product for oral and dental hygiene, hygiene article, cleaning composition, textile detergent composition, composition for scent dispensers, food, food supplement, pharmaceutical composition or crop protection composition.

In another particular embodiment the invention is directed to a method for imparting one of the following olfactory notes: herbal, incense; or any combination thereof to a perfume composition, body care composition, product for oral and dental hygiene, hygiene article, cleaning composition, textile detergent composition, composition for scent dispensers, food, food supplement, pharmaceutical composition or crop protection composition, which comprises including the ethyl ether of 2-hydroxy-1,4-cineole (compound (I) wherein R¹=CH₂CH₃) in a perfume composition, body care composition, product for oral and dental hygiene, hygiene article, cleaning composition, textile detergent composition, composition for scent dispensers, food, food supplement, pharmaceutical composition or crop protection composition.

Further general and preferred features of compositions A and B Composition A optionally and composition B mandatorily comprise at least one further component selected from the group consisting of aroma chemicals different from compounds (I) and (II), non-aroma chemical carriers, anti-oxidants and deodorant-active agents. In case of composition B the proviso applies that R¹ is not hydrogen if the composition comprises menthol.

The non-aroma chemical carriers, anti-oxidants and deodorant-active agents are of course also different from compounds (I) and (II).

The non-aroma chemical carrier is in particular selected from the group consisting of surfactants, oil components (emollients) and solvents.

Thus, in a preferred embodiment, the at least one further component is selected from the group consisting of aroma chemicals different from compounds (I) and (II), surfactants, oil components (emollients), solvents, anti-oxidants and deodorant-active agents.

The pleasant aroma, low volatility and excellent solubility make compounds (I) [and (II) in case of composition A] suitable components in compositions where a pleasing aroma is desirable.

Accordingly, composition A [if this is not a mixture consisting essentially of (I) and (II)] and composition B are preferably an aroma chemical composition, more preferably an odor composition and in particular a fragrance composition.

One embodiment of the invention is directed to a composition comprising a compound (I) or a stereoisomer thereof or a mixture of different stereoisomers thereof or a mixture of different compounds (I) as defined above [and in case of composition A additionally a compound (II) or a stereoisomer thereof or a mixture of different stereoisomers thereof] and at least one further component selected from the group consisting of oil components, solvents, anti-oxidants and deodorant-active agents.

One embodiment of the invention is directed to a composition comprising a compound (I) or a stereoisomer thereof or a mixture of different stereoisomers thereof or a mixture of different compounds (I) as defined above [and in case of composition A additionally a compound (II) or a stereoisomer thereof or a mixture of different stereoisomers thereof] and at least one aroma chemical.

One embodiment of the invention is directed to a composition comprising a compound (I) or a stereoisomer thereof or a mixture of different stereoisomers thereof or a mixture of different compounds (I) as defined above [and in case of composition A additionally a compound (II) or a stereoisomer thereof or a mixture of different stereoisomers thereof] and at least one non-aroma chemical carrier.

One embodiment of the invention is directed to a composition comprising a compound (I) or a stereoisomer thereof or a mixture of different stereoisomers thereof or a mixture of different compounds (I) as defined above [and in case of composition A additionally a compound (II) or a stereoisomer thereof or a mixture of different stereoisomers thereof] and at least one surfactant.

One embodiment of the invention is directed to a composition comprising a compound (I) or a stereoisomer thereof or a mixture of different stereoisomers thereof or a mixture of different compounds (I) as defined above [and in case of composition A additionally a compound (II) or a stereoisomer thereof or a mixture of different stereoisomers thereof] and at least one oil component.

One embodiment of the invention is directed to a composition comprising a compound (I) or a stereoisomer thereof or a mixture of different stereoisomers thereof or a mixture of different compounds (I) as defined above [and in case of composition A additionally a compound (II) or a stereoisomer thereof or a mixture of different stereoisomers thereof] and at least one solvent.

One embodiment of the invention is directed to a composition comprising a compound (I) or a stereoisomer thereof or a mixture of different stereoisomers thereof or a mixture of different compounds (I) as defined above [and in case of composition A additionally a compound (II) or a stereoisomer thereof or a mixture of different stereoisomers thereof] and at least one anti-oxidant.

One embodiment of the invention is directed to a composition comprising a compound (I) or a stereoisomer thereof or a mixture of different stereoisomers thereof or a mixture of different compounds (I) as defined above [and in case of composition A additionally a compound (II) or a stereoisomer thereof or a mixture of different stereoisomers thereof] and at least one deodorant-active agent.

The compound (I) [in case of composition A in admixture with compound (II)] can preferably be used in aroma compositions. In preferred embodiments, the aroma composition is an odor composition, i.e. a composition inducing an odor impression, and is in particular a fragrance composition, i.e. a composition inducing a pleasant odor.

Compositions A [if this is not a mixture consisting essentially of compounds (I) and (II))] and B can be selected from, but are not limited to, the group consisting of perfume compositions, body care compositions (including cosmetic compositions), products for oral and dental hygiene, hygiene articles, cleaning compositions (including dishwashing compositions), textile detergent compositions, compositions for scent dispensers, foods, food supplements, pharmaceutical compositions and crop protection compositions.

Aroma Chemicals [Different from Compounds (I) and (II)]

By virtue of the physical properties of the compounds (I) (and (II) in case of composition A), combinations of said compounds have particularly good, virtually universal solvent properties for and in aroma chemicals and other customary ingredients in aroma compositions such as, in particular, fragrance compositions. Therefore, the compound (I) (and (II) in case of composition A) is well combinable with aroma chemicals which are different from compounds (I) and (II), allowing, in particular, the creation of aroma compositions (preferably fragrance compositions) having novel advantageous sensory profiles. Especially, as already explained above, the combinations can boost the sensory profile of aroma chemicals (such as for example of fragrances) wherein said aroma chemicals are different from compounds (I) and (II).

The compositions A and B can comprise at least one aroma chemical that is different from compounds (I) and (II). Said at least one aroma chemical can for example be 1, 2, 3, 4, 5, 6, 7, 8 or more aroma chemicals, selected from the group consisting of: Geranyl acetate (3,7-dimethyl-2,6 octadien-1yl acetate), alpha-hexylcinnamaldehyde, 2-phenoxyethyl isobutyrate (Phenirat¹), dihydromyrcenol (2,6-dimethyl-7-octen-2-ol), methyl dihydrojasmonate (preferably with a content of cis isomer of more than 60 wt. %) (Hedione⁹, Hedione HC⁹), 4,6,6,7,8,8-hexamethyl-1,3,4,6,7,8-hexahydrocyclopenta[g]benzopyran (Galaxolid³), tetrahydrolinalool (3,7-dimethyloctan-3-ol), ethyllinalool, benzyl salicylate, 2-methyl-3-(4-tert-butylphenyl)propanal (Lysmeral²), cinnamyl alcohol, 4,7-methano-3a,4,5,6,7,7a-hexahydro-5-indenyl acetate and/or 4,7-methano-3a,4,5,6,7,7a-hexahydro-6-indenyl acetate (Herbaflorat¹), citronellol, citronellyl acetate, tetrahydrogeraniol, vanillin, linalyl acetate, styrolyl acetate (1-phenylethyl acetate), octahydro-2,3,8,8-tetramethyl-2-acetonaphthone and/or 2-acetyl-1,2,3,4,6,7,8-octahydro-2,3,8,8-tetramethylnaphthalene (Iso E Super³), hexyl salicylate, 4-tert-butylcyclohexyl acetate (Oryclone¹), 2-tert-butylcyclohexyl acetate (Agrumex HC¹), alpha-ionone (4-(2,2,6-trimethyl-2-cyclohexen-1-yl)-3-buten-2-one), n-alpha-methylionone, alpha-isomethylionone, coumarin, terpinyl acetate, 2-phenylethyl alcohol, 4-(4-hydroxy-4-methylpentyl)-3-cyclohexenecarboxaldehyde (Lyral³), alpha-amylcinnamaldehyde, ethylene brassylate, (E)- and/or (Z)-3-methyl-cyclopentadec-5-enone (Muscenon⁹), 15-pentadec-11-enolide and/or 15-pentadec-12-enolide (Globalide¹), 15-cyclopentadecanolide (Macrolide¹), 1-(5,6,7,8-tetrahydro-3,5,5,6,8,8-hexamethyl-2-naphthalenyl)ethanone (Tonalid¹⁰), 2-isobutyl-4-methyl-tetrahydro-2H-pyran-4-ol (Florol⁹), 2-ethyl-4-(2,2,3-trimethyl-3-cyclopenten-1-yl)-2-buten-1-ol (Sandolen¹), cis-3-hexenyl acetate, trans-3-hexenyl acetate, trans-2/cis-6-nonadienol, 2,4-dimethyl-3-cyclohexenecarboxaldehyde (Vertocitral¹), 2,4,4,7-tetra-methyloct-6-en-3-one (Claritone¹), 2,6-dimethyl-5-hepten-1-al (Melonal²), borneol, 3-(3-isopropylphenyl)butanal (Florhydral²), 2-methyl-3-(3,4-methylenedioxyphenyl)-propanal (Helional³), 3-(4-ethylphenyl)-2,2-dimethylpropanal (Florazon¹), 7-methyl-2H-1,5-benzodioxepin-3(4H)-one (Calone), 3,3,5-trimethylcyclohexyl acetate (preferably with a content of cis isomers of 70 wt. %) or more, 2,5,5-trimethyl-1,2,3,4,4a,5,6,7-octahydronaphthalen-2-ol (Ambrinol S¹), 3-(4-tert-butylphenyl)-propanal (Bourgeonal⁴), ethyl 2-methylpentanoate (Manzanate⁴), ethoxymethoxycyclododecane (Amberwood¹), 2,4-dimethyl-4,4a,5,9b-tetrahydroindeno[1,2-d][1,3]dioxine (Magnolan¹), (2-tert-butylcyclohexyl) acetate (Verdox³) and 3-[5,5,6-trimethylbicyclo[2.2.1]hept-2-yl]cyclohexan-1-ol (Sandela⁴). Within the context of the present invention, the aforementioned aroma chemical(s) are accordingly preferably combined with a compound of formula (I), or a mixture of different compounds (I), as described above.

Where trade names are given above, these refer to the following sources:
¹ trade name of Symrise GmbH, Germany;
² trade name of BASF SE;
³ trade name of International Flavors & Fragrances Inc., USA;
⁴ Givaudan AG, Switzerland;
⁹ trade name of Firmenich S.A., Switzerland;
¹⁰ trade name of PFW Aroma Chemicals B.V., the Netherlands.

A preferred embodiment of the invention relates to a composition comprising a compound (I) or a stereoisomer thereof or a mixture of different stereoisomers thereof or a mixture of different compounds (I) as defined above [and in case of composition A additionally a compound (II) or a stereoisomer thereof or a mixture of different stereoisomers thereof] and at least one aroma chemical selected from the group consisting of methyl benzoate, benzyl acetate, geranyl acetate, 2-isobutyl-4-methyltetrahydro-2H-pyran-4-ol and linalool.

A further embodiment of the invention relates to a composition comprising a compound (I) or a stereoisomer thereof or a mixture of different stereoisomers thereof or a mixture of different compounds (I) as defined above [and in case of composition A additionally a compound (II) or a stereoisomer thereof or a mixture of different stereoisomers thereof] and 2-isobutyl-4-methyltetrahydro-2H-pyran-4-ol.

A further embodiment of the invention relates to a composition comprising a compound (I) or a stereoisomer thereof or a mixture of different stereoisomers thereof or a mixture of different compounds (I) as defined [and in case of composition A additionally a compound (II) or a stereoisomer thereof or a mixture of different stereoisomers thereof] and methyl benzoate.

A preferred embodiment of the invention relates to a composition comprising a compound (I) or a stereoisomer thereof or a mixture of different stereoisomers thereof or a mixture of different compounds (I) as defined above [and in case of composition A additionally a compound (II) or a stereoisomer thereof or a mixture of different stereoisomers thereof] and at least one further aroma chemical selected from the group consisting of ethylvanillin, vanillin, 2,5-dimethyl-4-hydroxy-2H-furan-3-one (furaneol) or 3-hydroxy-2-methyl-4H-pyran-4-one (maltol).

Further aroma chemicals with which the compound (I) [in case of composition A in admixture with the compound (II)] can be combined to give a composition according to the invention can be found, e.g., in S. Arctander, Perfume and Flavor Chemicals, Vol. I and II, Montclair, N. J., 1969, self-published or K. Bauer, D. Garbe and H. Surburg, Common Fragrance and Flavor Materials, 4th Ed., Wiley- VCH, Weinheim 2001. Specifically, mention may be made of:
extracts from natural raw materials such as essential oils, concretes, absolutes, resins, resinoids, balsams, tinctures such as e.g.
ambergris tincture; amyris oil; angelica seed oil; angelica root oil; aniseed oil; valerian oil; basil oil; tree moss absolute; bay oil; mugwort oil; benzoin resin; bergamot oil; beeswax absolute; birch tar oil; bitter almond oil; savory oil; buchu leaf oil; cabreuva oil; cade oil; calmus oil; camphor oil; cananga oil; cardamom oil; cascarilla oil; cassia oil; cassia absolute; castoreum absolute; cedar leaf oil; cedar wood oil; cistus oil; citronella oil; lemon oil; copaiba balsam; copaiba balsam oil; coriander oil; costus root oil; cumin oil; cypress oil; davana oil; dill weed oil; dill seed oil; Eau de brouts absolute; oak moss absolute; elemi oil; tarragon oil; eucalyptus citriodora oil; eucalyptus oil; fennel oil; pine needle oil; galbanum oil; galbanum resin; geranium oil; grapefruit oil; guaiacwood oil; gurjun balsam; gurjun balsam oil; helichrysum absolute; helichrysum oil; ginger oil; iris root absolute; iris root oil; jasmine absolute; calmus oil; camomile oil blue; roman camomile oil; carrot seed oil; cascarilla oil; pine needle oil; spearmint oil; caraway oil; labdanum oil; labdanum absolute; labdanum resin; lavandin absolute; lavandin oil; lavender absolute; lavender oil; lemongrass oil; lovage oil; lime oil distilled; lime oil pressed; linalool oil; litsea cubeba oil; laurel leaf oil; mace oil; marjoram oil; mandarin oil; massoia bark oil; mimosa absolute; musk seed oil; musk tincture; clary sage oil; nutmeg oil; myrrh absolute; myrrh oil; myrtle oil; clove leaf oil; clove flower oil; neroli oil; olibanum absolute; olibanum oil; opopanax oil; orange blossom absolute; orange oil; origanum oil; palmarosa oil; patchouli oil; perilla oil; peru balsam oil; parsley leaf oil; parsley seed oil; petitgrain oil; peppermint oil; pepper oil; pimento oil; pine oil; pennyroyal oil; rose absolute; rose wood oil; rose oil; rosemary oil; Dalmatian sage oil; Spanish sage oil; sandalwood oil; celery seed oil; spike-lavender oil; star anise oil; styrax oil; tagetes oil; fir needle oil; tea tree oil; turpentine oil; thyme oil; tolubalsam; tonka absolute; tuberose absolute; vanilla extract; violet leaf absolute; verbena oil; vetiver oil; juniper berry oil; wine lees oil; wormwood oil; winter green oil; hyssop oil; civet absolute; cinnamon leaf oil; cinnamon bark oil, and fractions thereof, or ingredients isolated therefrom;
individual fragrances from the group of hydrocarbons, such as e.g. 3-carene; alpha-pinene; beta-pinene; alpha-terpinene; gamma-terpinene; p-cymene; bisabolene; camphene; caryophyllene; cedrene; farnesene; limonene; longifolene; myrcene; ocimene; valencene; (E,Z)-1,3,5-undecatriene; styrene; diphenylmethane;
the aliphatic alcohols such as e.g. hexanol; octanol; 3-octanol; 2,6-dimethylheptanol; 2-methyl-2-heptanol; 2-methyl-2-octanol; (E)-2-hexenol; (E)- and (Z)-3-hexenol; 1-octen-3-ol; mixture of 3,4,5,6,6-pentamethyl-3/4-hepten-2-ol and 3,5,6,6-tetramethyl-4-methyleneheptan-2-ol; (E,Z)-2,6-nonadienol; 3,7-dimethyl-7-methoxyoctan-2-ol; 9-decenol; 10-undecenol; 4-methyl-3-decen-5-ol;
the aliphatic aldehydes and acetals thereof such as e.g. hexanal; heptanal; octanal; nonanal; decanal; undecanal; dodecanal; tridecanal; 2-methyloctanal; 2-methylnonanal; (E)-2-hexenal; (Z)-4-heptenal; 2,6-dimethyl-5-heptenal; 10-undecenal; (E)-4-decenal; 2-dodecenal; 2,6,10-trimethyl-9-undecenal; 2,6,10-trimethyl-5,9-undecadienal; heptanal diethylacetal; 1,1-dimethoxy-2,2,5-trimethyl-4-hexene; citronellyloxyacetaldehyde; (E/Z)-1-(1-methoxypropoxy)-hex-3-ene; the aliphatic ketones and oximes thereof such as e.g. 2-heptanone; 2-octanone; 3-octanone; 2-nonanone; 5-methyl-3-heptanone; 5-methyl-3-heptanone oxime; 2,4,4,7-tetramethyl-6-octen-3-one; 6-methyl-5-hepten-2-one;
the aliphatic sulfur-containing compounds such as e.g. 3-methylthiohexanol; 3-methylthiohexyl acetate; 3-mercaptohexanol; 3-mercaptohexyl acetate; 3-mercaptohexyl butyrate; 3-acetylthiohexyl acetate; 1-menthene-8-thiol;
the aliphatic nitriles such as e.g. 2-nonenenitrile; 2-undecenenitrile; 2-tridecenenitrile; 3,12-tridecadienenitrile; 3,7-dimethyl-2,6-octadienenitrile; 3,7-dimethyl-6-octenenitrile; the esters of aliphatic carboxylic acids such as e.g. (E)- and (Z)-3-hexenyl formate; ethyl acetoacetate; isoamyl acetate; hexyl acetate; 3,5,5-trimethylhexyl acetate; 3-methyl-2-butenyl acetate; (E)-2-hexenyl acetate; (E)- and (Z)-3-hexenyl acetate; octyl acetate; 3-octyl acetate; 1-octen-3-yl acetate; ethyl butyrate; butyl butyrate; isoamyl butyrate; hexyl butyrate; (E)- and (Z)-3-hexenyl isobutyrate; hexyl crotonate; ethyl isovalerate; ethyl 2-methylpentanoate; ethyl hexanoate; allyl hexanoate; ethyl heptanoate; allyl heptanoate; ethyl octanoate; ethyl (E,Z)-2,4-decadienoate; methyl 2-octinate; methyl 2-noninate; allyl 2-isoamyloxy acetate; methyl-3,7-dimethyl-2,6-octadienoate; 4-methyl-2-pentyl crotonate;
the acyclic terpene alcohols such as e.g. geraniol; nerol; linalool; lavandulol; nerolidol; farnesol; tetrahydrolinalool; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2-ol; 3,7-dimethyl-4,6-octadien-3-ol; 3,7-dimethyl-1,5,7-octatrien-3-ol; 2,6-dimethyl-2,5,7-octatrien-1-ol; and the formates, acetates, propionates, isobutyrates, butyrates, isovalerates, pentanoates, hexanoates, crotonates, tiglinates and 3-methyl-2-butenoates thereof;
the acyclic terpene aldehydes and ketones such as e.g. geranial; neral; citronellal; 7-hydroxy-3,7-dimethyloctanal; 7-methoxy-3,7-dimethyloctanal; 2,6,10-trimethyl-9-undecenal; geranyl acetone; as well as the dimethyl- and diethylacetals of geranial, neral, 7-hydroxy-3,7-dimethyloctanal; the cyclic terpene alcohols such as e.g. menthol; isopulegol; alpha-terpineol; terpine-4-ol; menthan-8-ol; menthan-1-ol; menthan-7-ol; borneol; isoborneol; linalool oxide; nopol; cedrol; ambrinol; vetiverol; guajol; and the formates, acetates, propionates, isobutyrates, butyrates, isovalerates, pentanoates, hexanoates, crotonates, tiglinates and 3-methyl-2-butenoates thereof;
the cyclic terpene aldehydes and ketones such as e.g. menthone; isomenthone; 8-mercaptomenthan-3-one; carvone; camphor; fenchone; alpha-ionone; beta-ionone; alpha-n-methylionone; beta-n-methylionone; alpha-isomethylionone; beta-isomethylionone; alpha-irone; alpha-damascone; beta-damascone; beta-damascenone; delta-damascone; gamma-damascone; 1-(2,4,4-trimethyl-2-cyclohexen-1-yl)-2-buten-1-one; 1,3,4,6,7,8a-hexahydro-1,1,5,5-tetramethyl-2H-2,4a-methanonaphthalene-8(5H)-one; 2-methyl-4-(2,6,6-trimethyl-1-cyclohexen-1-yl)-2-butenal; nootkatone; dihydronootkatone; 4,6,8-megastigmatrien-3-one; alpha-sinensal; beta-sinensal; acetylated cedar wood oil (methyl cedryl ketone);
the cyclic alcohols such as e.g. 4-tert-butylcyclohexanol; 3,3,5-trimethylcyclohexanol; 3-isocamphylcyclohexanol; 2,6,9-trimethyl-Z2,Z5,E9-cyclododecatrien-1-ol; 2-isobutyl-4-methyltetrahydro-2H-pyran-4-ol;
the cycloaliphatic alcohols such as e.g. alpha-3,3-trimethylcyclohexylmethanol; 1-(4-isopropylcyclohexyl)ethanol; 2-methyl-4-(2,2,3-trimethyl-3-cyclopent-1-yl)butanol; 2-methyl-4-(2,2,3-trimethyl-3-cyclopent-1-yl)-2-buten-1-ol; 2-ethyl-4-(2,2,3-trimethyl-3-cyclopent-1-yl)-2-buten-1-ol; 3-methyl-5-(2,2,3-trimethyl-3-cyclopent-1-yl)pentan-2-ol; 3-methyl-5-(2,2,3-trimethyl-3-cyclopent-1-yl)-4-penten-2-ol; 3,3-dimethyl-5-(2,2,3-trimethyl-3-cyclopent-1-yl)-4-penten-2-ol; 1-(2,2,6-trimethylcyclohexyl)pentan-3-ol; 1-(2,2,6-trimethylcyclohexyl)hexan-3-ol;
the cyclic and cycloaliphatic ethers such as e.g. cineol; cedryl methyl ether; cyclododecyl methyl ether; 1,1-dimethoxycyclododecane; (ethoxymethoxy)cyclododecane; alpha-cedrene epoxide; 3a,6,6,9a-tetramethyldodecahydronaphtho[2,1-b]furan; 3a-ethyl-6,6,9a-trimethyldodecahydronaphtho[2,1-b]furan; 1,5,9-trimethyl-13-oxabicyclo-[10.1.0]trideca-4,8-diene; rose oxide; 2-(2,4-dimethyl-3-cyclohexen-1-yl)-5-methyl-5-(1-methylpropyl)-1,3-dioxane;
the cyclic and macrocyclic ketones such as e.g. 4-tert-butylcyclohexanone; 2,2,5-trimethyl-5-pentylcyclopentanone; 2-heptylcyclopentanone; 2-pentylcyclopentanone; 2-hydroxy-3-methyl-2-cyclopenten-1-one; 3-methyl-cis-2-penten-1-yl-2-cyclopenten-1-one; 3-methyl-2-pentyl-2-cyclopenten-1-one; 3-methyl-4-cyclopentadecenone; 3-methyl-5-cyclopentadecenone; 3-methylcyclopentadecanone; 4-(1-ethoxyvinyl)-3,3,5,5-tetramethylcyclohexanone; 4-tert-pentylcyclohexanone; 5-cyclohexadecen-1-one; 6,7-dihydro-1,1,2,3,3-pentamethyl-4(5H)-indanone; 8-cyclo-hexadecen-1-one; 7-cyclohexadecen-1-one; (7/8)-cyclohexadecen-1-one; 9-cycloheptadecen-1-one; cyclopentadecanone; cyclohexadecanone;
the cycloaliphatic aldehydes such as e.g. 2,4-dimethyl-3-cyclohexenecarbaldehyde; 2-methyl-4-(2,2,6-trimethylcyclohexen-1-yl)-2-butenal; 4-(4-hydroxy-4-methylpentyl)-3-cyclohexene carbaldehyde; 4-(4-methyl-3-penten-1-yl)-3-cyclohexenecarbaldehyde; the cycloaliphatic ketones such as e.g. 1-(3,3-dimethylcyclohexyl)-4-penten-1-one; 2,2-dimethyl-1-(2,4-dimethyl-3-cyclohexen-1-yl)-1-propanone; 1-(5,5-dimethyl-1-cyclohexen-1-yl)-4-penten-1-one; 2,3,8,8-tetramethyl-1,2,3,4,5,6,7,8-octahydro-2-naphthalenyl methyl ketone; methyl 2,6,10-trimethyl-2,5,9-cyclododecatrienyl ketone; tert-butyl (2,4-dimethyl-3-cyclohexen-1-yl) ketone;
the esters of cyclic alcohols such as e.g. 2-tert-butylcyclohexyl acetate; 4-tert-butylcyclohexyl acetate; 2-tert-pentylcyclohexyl acetate; 4-tert-pentylcyclohexyl acetate; 3,3,5-trimethylcyclohexyl acetate; decahydro-2-naphthyl acetate; 2-cyclopentylcyclopentyl crotonate; 3-pentyltetrahydro-2H-pyran-4-yl acetate; decahydro-2,5,5,8a-tetramethyl-2-naphthyl acetate; 4,7-methano-3a,4,5,6,7,7a-hexahydro-5 or 6-indenyl acetate; 4,7-methano-3a,4,5,6,7,7a-hexahydro-5 or 6-indenyl propionate; 4,7-methano-3a,4,5,6,7,7a-hexahydro-5 or 6-indenyl isobutyrate; 4,7-methanooctahydro-5 or 6-indenyl acetate;
the esters of cycloaliphatic alcohols such as e.g. 1-cyclohexylethyl crotonate;
the esters of cycloaliphatic carboxylic acids such as e.g. allyl 3-cyclohexylpropionate; allyl cyclohexyloxyacetate; cis- and trans-methyl dihydrojasmonate; cis- and transmethyl jasmonate; methyl 2-hexyl-3-oxocyclopentanecarboxylate; ethyl 2-ethyl-6,6-dimethyl-2-cyclohexenecarboxylate; ethyl 2,3,6,6-tetramethyl-2-cyclohexenecarboxylate; ethyl 2-methyl-1,3-dioxolane-2-acetate;
the araliphatic alcohols such as e.g. benzyl alcohol; 1-phenylethyl alcohol, 2-phenylethyl alcohol, 3-phenylpropanol; 2-phenylpropanol; 2-phenoxyethanol; 2,2-dimethyl-3-phenylpropanol; 2,2-dimethyl-3-(3-methylphenyl)propanol; 1,1-dimethyl-2-phenylethyl alcohol; 1,1-dimethyl-3-phenylpropanol; 1-ethyl-1-methyl-3-phenylpropanol; 2-methyl-5-phenylpentanol; 3-methyl-5-phenylpentanol; 3-phenyl-2-propen-1-ol; 4-methoxybenzyl alcohol; 1-(4-isopropylphenyl)ethanol;
the esters of araliphatic alcohols and aliphatic carboxylic acids such as e.g. benzyl acetate; benzyl propionate; benzyl isobutyrate; benzyl isovalerate; 2-phenylethyl acetate; 2-phenylethyl propionate; 2-phenylethyl isobutyrate; 2-phenylethyl isovalerate; 1-phenylethyl acetate; alpha-trichloromethylbenzyl acetate; alpha,alpha-dimethylphenylethyl acetate; alpha,alpha-dimethylphenylethyl butyrate; cinnamyl acetate; 2-phenoxyethyl isobutyrate; 4-methoxybenzyl acetate;
the araliphatic ethers such as e.g. 2-phenylethyl methyl ether; 2-phenylethyl isoamyl ether; 2-phenylethyl 1-ethoxyethyl ether; phenylacetaldehyde dimethyl acetal; phenylacetaldehyde diethyl acetal; hydratropaaldehyde dimethyl acetal; phenylacetaldehyde glycerol acetal; 2,4,6-trimethyl-4-phenyl-1,3-dioxane; 4,4a,5,9b-tetrahydroindeno[1,2-d]-m-dioxine; 4,4a,5,9b-tetrahydro-2,4-dimethylindeno[1,2-d]-m-dioxine;
the aromatic and araliphatic aldehydes such as e.g. benzaldehyde; phenylacetaldehyde; 3-phenylpropanal; hydratropaaldehyde; 4-methylbenzaldehyde; 4-methylphenylacetaldehyde; 3-(4-ethylphenyl)-2,2-dimethylpropanal; 2-methyl-3-(4-isopropylphenyl)propanal; 2-methyl-3-(4-tert-butylphenyl)propanal; 2-methyl-3-(4-isobutylphenyl)propanal; 3-(4-tert-butylphenyl)propanal; cinnamaldehyde; alpha-butylcinnamaldehyde; alpha-amylcinnamaldehyde; alpha-hexylcinnamaldehyde; 3-methyl-5-phenylpentanal; 4-methoxybenzaldehyde; 4-hydroxy-3-methoxybenzaldehyde; 4-hydroxy-3-ethoxybenzaldehyde; 3,4-methylenedioxybenzaldehyde; 3,4-dimethoxybenzaldehyde; 2-methyl-3-(4-methoxyphenyl)propanal; 2-methyl-3-(4-methylenedioxyphenyl)propanal;
the aromatic and araliphatic ketones such as e.g. acetophenone;
4-methylacetophenone; 4-methoxyacetophenone; 4-tert-butyl-2,6-dimethylacetophenone; 4-phenyl-2-butanone; 4-(4-hydroxyphenyl)-2-butanone; 1-(2-naphthalenyl)-ethanone; 2-benzofuranylethanone; (3-methyl-2-benzofuranyl)ethanone; benzophenone; 1,1,2,3,3,6-hexamethyl-5-indanyl methyl ketone; 6-tert-butyl-1,1-dimethyl-4-indanyl methyl ketone; 1-[2,3-dihydro-1,1,2,6-tetramethyl-3-(1-methylethyl)-1H-5-indenyl]ethanone; 5′,6′,7′,8′-tetrahydro-3′,5′,5′,6′,8′,8′-hexamethyl-2-acetonaphthone; the aromatic and araliphatic carboxylic acids and esters thereof such as e.g. benzoic acid; phenylacetic acid; methyl benzoate; ethyl benzoate; hexyl benzoate; benzyl benzoate; methyl phenylacetate; ethyl phenylacetate; geranyl phenylacetate; phenylethyl phenylacetate; methyl cinnamate; ethyl cinnamate; benzyl cinnamate; phenylethyl cinnamate; cinnamyl cinnamate; allyl phenoxyacetate; methyl salicylate; isoamyl salicylate; hexyl salicylate; cyclohexyl salicylate; cis-3-hexenyl salicylate; benzyl salicylate; phenylethyl salicylate; methyl 2,4-dihydroxy-3,6-dimethylbenzoate; ethyl 3-phenylglycidate; ethyl 3-methyl-3-phenylglycidate;
the nitrogen-containing aromatic compounds such as e.g. 2,4,6-trinitro-1,3-dimethyl-5-tert-butylbenzene; 3,5-dinitro-2,6-dimethyl-4-tert-butylacetophenone; cinnamonitrile; 3-methyl-5-phenyl-2-pentenonitrile; 3-methyl-5-phenylpentanonitrile; methyl anthranilate; methyl-N-methylanthranilate; Schiff bases of methyl anthranilate with 7-hydroxy-3,7-dimethyloctanal, 2-methyl-3-(4-tert-butylphenyl)propanal or 2,4-dimethyl-3-cyclohexenecarbaldehyde; 6-isopropylquinoline; 6-isobutylquinoline; 6-secbutylquinoline; 2-(3-phenylpropyl)pyridine; indole; skatole; 2-methoxy-3-isopropylpyrazine; 2-isobutyl-3-methoxypyrazine;
the phenols, phenyl ethers and phenyl esters such as e.g. estragole; anethole; eugenol; eugenyl methyl ether; isoeugenol; isoeugenyl methyl ether; thymol; carvacrol; diphenyl ether; beta-naphthyl methyl ether; beta-naphthyl ethyl ether; beta-naphthyl isobutyl ether; 1,4-dimethoxybenzene; eugenyl acetate; 2-methoxy-4-methylphenol; 2-ethoxy-5-(1-propenyl)phenol; p-cresyl phenylacetate;
the heterocyclic compounds such as e.g. 2,5-dimethyl-4-hydroxy-2H-furan-3-one; 2-ethyl-4-hydroxy-5-methyl-2H-furan-3-one; 3-hydroxy-2-methyl-4H-pyran-4-one; 2-ethyl-3-hydroxy-4H-pyran-4-one;
the lactones such as e.g. 1,4-octanolide; 3-methyl-1,4-octanolide; 1,4-nonanolide; 1,4-decanolide; 8-decen-1,4-olide; 1,4-undecanolide; 1,4-dodecanolide; 1,5-decanolide; 1,5-dodecanolide; 4-methyl-1,4-decanolide; 1,15-pentadecanolide; cis- and trans-11-pentadecen-1,15-olide; cis- and trans-12-pentadecen-1,15-olide; 1,16-hexadecanolide; 9-hexadecen-1,16-olide; 10-oxa-1,16-hexadecanolide; 11-oxa-1,16-hexadecanolide; 12-oxa-1,16-hexadecanolide; ethylene 1,12-dodecanedioate; ethylene 1,13-tridecanedioate; coumarin; 2,3-dihydrocoumarin; octahydrocoumarin.

The aroma chemicals different from compound (I) and (II) in compositions A and B are preferably selected from the group consisting of geranyl acetate, alpha-hexylcinnamaldehyde, 2-phenoxyethyl isobutyrate, dihydromyrcenol, methyl dihydrojasmonate (preferably with a content of cis isomer of more than 60 wt. %), 4,6,6,7,8,8-hexamethyl-1,3,4,6,7,8-hexahydrocyclopenta[g]benzopyran, tetrahydrolinalool, ethyllinalool, benzyl salicylate, 2-methyl-3-(4-tert-butylphenyl)propanal, cinnamyl alcohol, 4,7-methano-3a,4,5,6,7,7a-hexahydro-5-indenyl acetate, 4,7-methano-3a,4,5,6,7,7a-hexahydro-6-indenyl acetate, citronellol, citronellyl acetate, tetrahydrogeraniol, vanillin, linalyl acetate, styrolyl acetate, octahydro-2,3,8,8-tetramethyl-2-acetonaphthone, 2-acetyl-1,2,3,4,6,7,8-octahydro-2,3,8,8-tetramethylnaphthalene, hexyl salicylate, 4-tert-butylcyclohexyl acetate, 2-tert-butylcyclohexyl acetate, alpha-ionone, n-alpha-methylionone, alpha-isomethylionone, coumarin, terpinyl acetate, 2-phenylethyl alcohol, 4-(4-hydroxy-4-methylpentyl)-3-cyclohexenecarboxaldehyde, alpha-amylcinnamaldehyde, ethylene brassylate, (E)-3-methylcyclopentadec-5-enone, (Z)-3-methylcyclopentadec-5-enone, 15-pentadec-11-enolide, 15-pentadec-12-enolide, 15-cyclopentadecanolide, 1-(5,6,7,8-tetrahydro-3,5,5,6,8,8-hexamethyl-2-naphthalenyl)ethanone, 2-isobutyl-4-methyltetrahydro-2H-pyran-4-ol, 2-ethyl-4-(2,2,3-trimethyl-3-cyclopenten-1-yl)-2-buten-1-ol, cis-3-hexenyl acetate, trans-3-hexenyl acetate, trans-2/cis-6-nonadienol, 2,4-dimethyl-3-cyclohexenecarboxaldehyde, 2,4,4,7-tetramethyloct-6-en-3-one, 2,6-dimethyl-5-hepten-1-al, borneol, 3-(3-isopropylphenyl)butanal, 2-methyl-3-(3,4-methylenedioxyphenyl)propanal, 3-(4-ethylphenyl)-2,2-dimethylpropanal, 7-methyl-2H-1,5-benzodioxepin-3(4H)-one, 3,3,5-trimethylcyclohexyl acetate (preferably with a content of cis isomers of 70 wt. % or more), 2,5,5-trimethyl-1,2,3,4,4a,5,6,7-octahydronaphthalen-2-ol, 3-(4-tert-butylphenyl)-propanal, ethyl 2-methylpentanoate, ethoxymethoxycyclododecane, 2,4-dimethyl-4,4a,5,9b-tetrahydroindeno[1,2-d][1,3]dioxine, (2-tert-butylcyclohexyl) acetate, 3-[5,5,6-trimethylbicyclo[2.2.1]hept-2-yl]cyclohexan-1-ol, menthone, isomenthone, carvone, camphor, beta-ionone, beta-n-methylionone, beta-isomethylionone, alpha-irone, alpha-damascone, beta-damascone, beta-damascenone, delta-damascone, acetylated cedar wood oil, and mixtures thereof.

### Non-Aroma Chemical Carriers

A further embodiment of the invention is directed to a composition comprising a compound (I) or a stereoisomer thereof or a mixture of different stereoisomers thereof or a mixture of different compounds (I) as defined above [and in case of composition A additionally a compound (II) or a stereoisomer thereof or a mixture of different stereoisomers thereof] and at least one non-aroma chemical carrier.

The at least one non-aroma chemical carrier can be a compound, a mixture of compounds or other additives, which has/have no or no noteworthy sensory properties. The non-aroma chemical carrier can serve for the dilution and/or the fixing of the compound (I) (and (II) in case of composition A) and optionally the at least one further aroma chemical, as defined above, or any other component, if comprised in the composition.

A further embodiment of the invention is directed to a composition comprising a compound (I) or a stereoisomer thereof or a mixture of different stereoisomers thereof or a mixture of different compounds (I) as defined above [and in case of composition A additionally a compound (II) or a stereoisomer thereof or a mixture of different stereoisomers thereof] and at least one non-aroma chemical carrier selected from the group consisting of solvents, surfactants and oil components.

According to preferred embodiments of the present invention, said non-aroma chemical carrier(s) is/are selected from the solvents, surfactants and oil components listed below.

One embodiment of the invention is directed to a composition comprising a compound (I) or a stereoisomer thereof or a mixture of different stereoisomers thereof or a mixture of different compounds (I) as defined above [and in case of composition A additionally a compound (II) or a stereoisomer thereof or a mixture of different stereoisomers thereof] and at least one solvent.

In the context of the present invention, a “solvent” serves for the dilution of the compound(s) (I) (and (II) in case of composition A) to be used according to the invention and/or any further component of the composition without having its own aroma.

The one or more solvent(s) can be present in the composition in amount of 0.01 to 99 wt. % based on the composition. In a preferred embodiment of the invention, the composition comprises 0.1 to 90 wt. %, preferably 0.5 to 80 wt. % of solvent(s) based on the total weight of the composition. The amount of solvent(s) can be chosen depending on the composition. In one embodiment of the invention, the composition comprises 0.05 to 10 wt. %, preferably 0.1 to 5 wt. %, more preferably 0.2 to 3 wt. % based on the total weight of the composition. In one embodiment of the invention, the composition comprises 20 to 70 wt. %, preferably 25 to 50 wt. % of solvent(s) based on the total weight of the composition.

Preferred solvents are ethanol, isopropanol, dipropylene glycol (DPG), propylene glycol, 1,2-butylene glycol, glycerol, diethylene glycol monoethyl ether, diethyl phthalate (DEP), isopropyl myristate (IPM), triethyl citrate (TEC), and benzyl benzoate (BB). Especially preferred solvents are selected from the group consisting of ethanol, propylene glycol, dipropylene glycol, triethyl citrate, benzyl benzoate and isopropyl myristate.

In another preferred embodiment of the invention, the solvent is selected from the group consisting of ethanol, isopropanol, diethylene glycol monoethyl ether, glycerol, propylene glycol, 1,2-butylene glycol, dipropylene glycol, triethyl citrate and isopropyl myristate.

According to a further embodiment, the compound (I) or the stereoisomer thereof or the mixture of different stereoisomers thereof or the mixture of different compounds (I) as defined above (in case of composition A in admixture with the compound (II) or a stereoisomer thereof or a mixture of different stereoisomers thereof), is used according to the present invention in surfactant-containing compositions. Due to its characteristic fragrance property and its substantivity, tenacity as well as stability, the compound (I) or the stereoisomer thereof or the mixture of different stereoisomers thereof or the mixture of different compounds (I) as defined above (in case of composition A in admixture with the compound (II) or a stereoisomer thereof or a mixture of different stereoisomers thereof) can especially be used to provide an odor, preferably a fragrance impression to surfactant-containing compositions such as, for example, cleaners (in particular laundry care products and all-purpose cleaners). It can preferably be used to impart a long-lasting clean note to a surfactant comprising composition.

One embodiment of the invention is therefore directed to a composition comprising a compound (I) or a stereoisomer thereof or a mixture of different stereoisomers thereof or a mixture of different compounds (I) as defined above [and in case of composition A additionally a compound (II) or a stereoisomer thereof or a mixture of different stereoisomers thereof] and at least one surfactant.

The surfactant(s) may be selected from anionic, non-ionic, cationic and/or amphoteric or zwitterionic surfactants. Surfactant-containing compositions, such as for example shower gels, foam baths, shampoos, etc., preferably contain at least one anionic surfactant.

The compositions according to the invention usually contain the surfactant(s), in the aggregate, in an amount of 0 to 40 wt. %, preferably 0 to 20 wt. %, more preferably 0.1 to 15 wt. %, and particularly 0.1 to 10 wt. %, based on the total weight of the composition. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partly oxidized alk(en)yl oligoglycosides or glucuronic acid derivatives, fatty acid-N-alkyl glucamides, protein hydrolyzates (particularly wheat-based vegetable products), polyol fatty acid esters, sugar esters, sorbitan esters, polysorbates and amine oxides. If the nonionic surfactants contain polyglycol ether chains, they may have a conventional homolog distribution, although they preferably have a narrow-range homolog distribution.

Zwitterionic surfactants are surface-active compounds which contain at least one quaternary ammonium group and at least one —COOH⁽⁻⁾ or —SO₃⁽⁻⁾ group in the molecule. Particularly suitable zwitterionic surfactants are the so-called betaines, such as the N-alkyl-N,N-dimethyl ammonium glycinates, for example, cocoalkyl dimethyl ammonium glycinate, N-acylaminopropyl-N,N-dimethyl ammonium glycinates, for example, cocoacylaminopropyl dimethyl ammonium glycinate, and 2-alkyl-3-carboxymethyl-3-hydroxyethyl imidazolines, containing 8 to 18 carbon atoms in the alkyl or acyl group, and cocoacylaminoethyl hydroxyethyl carboxymethyl glycinate. The fatty acid amide derivative known under the CTFA name of Cocamidopropyl Betaine is particularly preferred.

Ampholytic surfactants are also suitable, particularly as co-surfactants. Ampholytic surfactants are surface-active compounds which, in addition to a C₈ to C₁₈ alkyl or acyl group, contain at least one free amino group and at least one —COOH— or —SO₃H— group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group. Particularly preferred ampholytic surfactants are N-cocoalk-ylaminopropionate, cocoacylaminoethyl aminopropionate and acyl sarcosine.

Anionic surfactants are characterized by a water-solubilizing anionic group such as, for example, a carboxylate, sulfate, sulfonate or phosphate group and a lipophilic group. Dermatologically safe anionic surfactants are known to the practitioner in large numbers from relevant textbooks and are commercially available. They are, in particular, alkyl sulfates in the form of their alkali metal, ammonium or alkanolammonium salts, alkylether sulfates, alkylether carboxylates, acyl isethionates, acyl sarcosinates, acyl taurines containing linear C₁₂-C₁₈ alkyl or acyl groups and sulfosuccinates and acyl glutamates in the form of their alkali metal or ammonium salts.

Particularly suitable cationic surfactants are quaternary ammonium compounds, preferably ammonium halides, more especially chlorides and bromides, such as alkyl trimethyl ammonium chlorides, dialkyl dimethyl ammonium chlorides and trialkyl methyl ammonium chlorides, for example, cetyl trimethyl ammonium chloride, stearyl trim ethyl ammonium chloride, distearyl dimethyl ammonium chloride, lauryl dimethyl ammonium chloride, lauryl dimethyl benzyl ammonium chloride and tricetyl methyl ammonium chloride. In addition, the readily biodegradable quaternary ester compounds, such as, for example, the dialkyl ammonium methosulfates and methyl hydroxyalkyl dialkoyloxyalkyl ammonium methosulfates marketed under the name of Stepantexe and the corresponding products of the Dehyquart® series, may be used as cationic surfactants. “Esterquats” are generally understood to be quaternized fatty acid triethanolamine ester salts. They can provide the compositions with particular softness. They are known substances which are prepared by the relevant methods of organic chemistry. Other cationic surfactants suitable for use in accordance with the invention are the quaternized protein hydrolyzates.

One embodiment of the invention is directed to a composition comprising a compound (I) or a stereoisomer thereof or a mixture of different stereoisomers thereof or a mixture of different compounds (I) as defined above [and in case of composition A additionally a compound (II) or a stereoisomer thereof or a mixture of different stereoisomers thereof] and at least one oil component.

The oil components are typically present in an amount of 0.1 to 80 wt. %, preferably 0.5 to 70 wt. %, more preferably 1 to 60 wt. %, even more preferably 1 to 50 wt. %, in particular 1 to 40 wt. %, more particularly 5 to 25 wt. % and specifically 5 to 15 wt. % based on the total weight of the composition.

The oil components may be selected, for example, from Guerbet alcohols based on fatty alcohols containing 6 to 18, preferably 8 to 10, carbon atoms and other additional esters, such as myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of C₁₈-C₃₈ alkyl-hydroxycarboxylic acids with linear or branched C₆-C₂₂ fatty alcohols, more especially dioctyl malate, esters of linear and/or branched fatty acids with polyhydric alcohols (for example propylene glycol, dimer dial or trimer triol), triglycerides based on C₆-C₁₀ fatty acids, liquid mono-, di- and triglyceride mixtures based on C₆-C₁₈ fatty acids, esters of C₆-C₂₂ fatty alcohols and/or Guerbet alcohols with aromatic carboxylic acids, more particularly benzoic acid, esters of dicarboxylic acids with polyols containing 2 to 10 carbon atoms and 2 to 6 hydroxyl groups, vegetable oils, branched primary alcohols, substituted cyclohexanes, linear and branched C₆-C₂₂ fatty alcohol carbonates such as, for example, dicaprylyl carbonate (Cetiol® CC), Guerbet carbonates based on fatty alcohols containing 6 to 18, preferably 8 to 10, carbon atoms, esters of benzoic acid with linear and/or branched C₆ to C₂₂ alcohols (for example Finsolv® TN), linear or branched, symmetrical or nonsymmetrical dialkyl ethers containing 6 to 22 carbon atoms per alkyl group such as, for example, dicaprylyl ether (Cetiol® OE), ring opening products of epoxidized fatty acid esters with polyols and hydrocarbons or mixtures thereof.

### Anti-Oxidants

One embodiment of the invention is directed to a composition comprising a compound (I) or a stereoisomer thereof or a mixture of different stereoisomers thereof or a mixture of different compounds (I) as defined above [and in case of composition A additionally a compound (II) or a stereoisomer thereof or a mixture of different stereoisomers thereof] and at least one anti-oxidant.

Anti-oxidants are able to inhibit or prevent the undesired changes in the compositions to be protected caused by oxygen effects and other oxidative processes. The effect of the anti-oxidants consists in most cases in them acting as free-radical scavengers for the free radicals which arise during autoxidation.

Anti-oxidants can for example be selected from the group consisting of
amino acids (for example glycine, alanine, arginine, serine, threonine, histidine, tyrosine, tryptophan) and derivatives thereof,
imidazoles (e.g. urocanic acid) and derivatives thereof,
peptides, such as D,L-carnosine, D-carnosine, L-carnosine (=β-Alanyl-L-histidin) and derivatives thereof
carotenoids, carotenes (e.g. alpha-carotene, beta-carotene, lycopene, lutein) or derivatives thereof,
chlorogenic acid and derivatives thereof,
lipoic acid and derivatives thereof (for example dihydrolipoic acid),
auro-thioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, gamma-linoleyl, cholesteryl and glyceryl esters thereof) and salts thereof,
dilauryl thiodipropionate, distearyl thiodipropionate, thiodipropionic acid and derivatives thereof (esters, ethers, peptides, lipids, nucleotides, nucleosides and salts),
sulfoximine compounds (for example buthionine sulfoximines, homocysteine sulfoximine, buthionine sulfones, penta-, hexa-, heptathionine sulfoximine)
(metal) chelating agents (e.g. alpha-hydroxy fatty acids, palmitic acid, phytic acid, lactoferrin),
alpha-hydroxy acids (for example citric acid, lactic acid, malic acid),
humic acid, bile acid, bile extracts, bilirubin, biliverdin, boldin (=alkaloid from the plant Peumus boldus, boldo extract,
EDTA, EGTA and derivatives thereof,
unsaturated fatty acids and derivatives thereof (e.g. gamma-linolenic acid, linoleic acid, oleic acid),
folic acid and derivatives thereof,
ubiquinone and ubiquinol and derivatives thereof,
vitamin C and derivatives (for example ascorbyl palmitate, Mg ascorbyl phosphate, ascorbyl acetate),
tocopherols and derivatives (for example vitamin E acetate),
vitamin A and derivatives (for example vitamin A palmitate),
coniferyl benzoate of gum benzoin, rutic acid and derivatives thereof, alpha-glycosylrutin, ferulic acid, furfurylideneglucitol,
butylhydroxytoluene (BHT), butylhydroxyanisole (BHA)
nordihydroguaiacic acid, nordihydroguaiaretic acid, trihydroxybutyrophenone, uric acid and derivatives thereof, mannose and derivatives thereof,
superoxide dismutase
zinc and derivatives thereof (for example ZnO, ZnSO4),
selenium and derivatives thereof (for example selenomethionine) and
stilbenes and derivatives thereof (e.g. stilbene oxide, trans-stilbene oxide)

In a preferred embodiment, the anti-oxidant is selected from the group consisting of pentaerythrityl, tetra-di-t-butyl hydroxyhydrocinnamate, nordihydroguaiaretic acid, ferulic acid, resveratrol, propyl gallate, butylhydroxytoluene (BHT), butylhydroxyanisole (BHA), ascorbyl palmitate and tocopherol.

The compositions according to the invention can comprise the anti-oxidants in an amount of 0.001 to 25 wt.-%, preferably 0.005 to 10 wt.-%, preferably 0.01 to 8 wt.-%, preferably 0.025 to 7 wt.-%, preferably 0.05 to 5 wt.-%, based on the total weight of the composition.

### Deodorant-Active Agents

One embodiment of the invention is directed to a composition comprising a compound (I) or a stereoisomer thereof or a mixture of different stereoisomers thereof or a mixture of different compounds (I) as defined [and in case of composition A additionally a compound (II) or a stereoisomer thereof or a mixture of different stereoisomers thereof] and at least one deodorant-active agent.

The compound (I) or the stereoisomer thereof or the mixture of different stereoisomers thereof or the mixture of different compounds (I) as defined above (in case of composition A in admixture with the compound (II) or the stereoisomer thereof or a mixture of different stereoisomers thereof) can be used to impart a clean, long-lasting note to deodorizing compositions as well as to the skin treated with such compositions.

Deodorizing compositions (deodorants and antiperspirants) counteract, mask or eliminate body odors. Body odors are formed through the action of skin bacteria on apocrine perspiration which results in the formation of unpleasant-smelling degradation products.

In a preferred embodiment of the invention, the at least one deodorant-active agent is selected from the groups consisting of anti-perspirants, esterase inhibitors and antibacterial agents.

Suitable antiperspirants can be selected from the group consisting of salts of aluminium, zirconium or zinc. Examples are aluminium chloride, aluminium chlorohydrate, aluminium dichlorohydrate, aluminium sesquichlorohydrate and complex compounds thereof, for example with 1,2-propylene glycol, aluminium hydroxyallantoinate, aluminium chloride tartrate, aluminium zirconium trichlorohydrate, aluminium zirconium tetrachlorohydrate, aluminium zirconium pentachlorohydrate and complex compounds thereof, for example with amino acids, such as glycine. Aluminium chlorohydrate, aluminium zirconium tetrachlorohydrate, aluminium zirconium pentachlorohydrate and complex compounds thereof are preferably used.

In a preferred embodiment of the invention the compositions comprise at least one antiperspirant selected from the group consisting aluminium chloride, aluminium chlorohydrate, aluminium dichlorohydrate, aluminium sesquichlorohydrate, aluminium hydroxyallantoinate, aluminium chloride tartrate, aluminium zirconium trichlorohydrate, aluminium zirconium tetrachlorohydrate and aluminium zirconium pentachlorohydrate

The compositions according to the invention can comprise the antiperspirants in an amount of 1 to 50, preferably 5 to 30 and more particularly 10 to 25 wt.-%, based on the solids content of the composition.

Where perspiration is present in the underarm region, extracellular enzymes-esterases, mainly proteases and/or lipases are formed by bacteria and split the esters present in the perspiration, releasing odors in the process. Suitable esterase inhibitors are for example trialkyl citrates, such as trimethyl citrate, tripropyl citrate, triisopropyl citrate, tributyl citrate and, in particular, triethyl citrate. Esterase inhibitors inhibit enzyme activity and thus reduce odor formation. The free acid is probably released by the cleavage of the citric acid ester and reduces the pH value of the skin to such an extent that the enzymes are inactivated by acylation. Other esterase inhibitors are sterol sulfates or phosphates such as, for example, lanosterol, cholesterol, campesterol, stigmasterol and sitosterol sulfate or phosphate, dicarboxylic acids and esters thereof, for example glutaric acid, glutaric acid monoethyl ester, glutaric acid diethyl ester, adipic acid, adipic acid monoethyl ester, adipic acid diethyl ester, malonic acid and malonic acid diethyl ester, hydroxycarboxylic acids and esters thereof, for example citric acid, malic acid, tartaric acid or tartaric acid diethyl ester, and zinc glycinate.

In a preferred embodiment of the invention the compositions comprise at least one esterase inhibitor selected from the group consisting of trimethyl citrate, tripropyl citrate, triisopropyl citrate, tributyl citrate triethyl citrate, lanosterol, cholesterol, campesterol, stigmasterol, sitosterol sulfate, sitosterol phosphate, glutaric acid, glutaric acid monoethyl ester, glutaric acid diethyl ester, adipic acid, adipic acid monoethyl ester, adipic acid diethyl ester, malonic acid, malonic acid diethyl ester, citric acid, malic acid, tartaric acid, tartaric acid diethyl ester and zinc glycinate.

The compositions according to the invention can comprise the esterase inhibitors in amounts of 0.01 to 20, preferably 0.1 to 10 and more particularly 0.5 to 5 wt.-%, based on the solids content of the composition.

The term “anti-bacterial agents” as used herein encompasses substances which have bactericidal and/or bacteriostatic properties. Typically these substances act against gram-positive bacteria such as, for example, 4-hydroxybenzoic acid and salts and esters thereof, N-(4-chlorophenyl)-N′-(3,4-dichlorophenyl)-urea, 2,4,4′-trichloro-2′-hydroxydiphenylether (triclosan), 4-chloro-3,5-dimethylphenol, 2,2′-methylene-bis-(6-bromo-4-chlorophenol), 3-methyl-4-(1-methylethyl)-phenol, 2-benzyl-4-chlorophenol, 3-(4-chlorophenoxy)-propane-1,2-diol, 3-iodo-2-propinyl butyl carbamate, chlorhexidine, 3,4,4′-trichlorocarbanilide (TTC), phenoxyethanol, glycerol monocaprate, glycerol monocaprylate, glycerol monolaurate (GML), diglycerol monocaprate (DMC), salicylic acid-N-alkylamides such as, for example, salicylic acid-n-octyl amide or salicylic acid-ndecyl amide.

In a preferred embodiment the antibacterial agent is selected from the group consisting of chitosan, phenoxyethanol, 5-chloro-2-(2,4-dichlorophenoxy)-phenol, 4-hydroxybenzoic acid and salts and esters thereof, N-(4-chlorophenyl)-N′-(3,4-dichlorophenyl)-urea, 2,4,4′-trichloro-2′-hydroxydiphenylether (triclosan), 4-chloro-3,5-dimethylphenol, 2,2′-methylene-bis-(6-bromo-4-chlorophenol), 3-methyl-4-(1-methylethyl)-phenol, 2-benzyl-4-chlorophenol, 3-(4-chlorophenoxy)-propane-1,2-diol, 3-iodo-2-propinyl butyl carbamate, chlorhexidine, 3,4,4′-trichlorocarbanilide (TTC), phenoxyethanol, glycerol monocaprate, glycerol monocaprylate, glycerol monolaurate (GML), diglycerol monocaprate (DMC), salicylic acid-N-alkylamides.

The compositions according to the invention can comprise the antibacterial agents in amounts of 0.01 to 5 wt. % and preferably 0.1 to 2 wt.-%, based on the solids content of the composition.

The compositions according to the invention can further comprise one or more substances, such as, for example: preservatives, abrasives, anti-acne agents, agents to combat skin aging, anti-cellulite agents, antidandruff agents, anti-inflammatory agents, irritation-preventing agents, irritation-alleviating agents, astringents, sweat-inhibiting agents, antiseptics, anti-statics, binders, buffers, carrier materials, chelating agents, cell stimulants, care agents, hair removal agents, emulsifiers, enzymes, essential oils, fibers, film formers, fixatives, foam formers, foam stabilizers, substances for preventing foaming, foam boosters, fungicides, gelling agents, gel-forming agents, hair care agents, hair shaping agents, hair smoothing agents, moisture-donating agents, moisturizing substances, humectant substances, bleaching agents, strengthening agents, stain removal agents, optical brighteners, impregnating agents, soil repellents, friction-reducing agents, lubricants, moisturizing creams, ointments, opacifiers, plasticizers, covering agents, polish, shine agents, polymers, powders, proteins, refatting agents, exfoliating agents, silicones, skin-calming agents, skin-cleansing agents, skin care agents, skin-healing agents, skin lightening agents, skin-protective agents, skin-softening agents, cooling agents, skin-cooling agents, warming agents, skin-warming agents, stabilizers, UV-absorbent agents, UV filters, fabric softeners, suspending agents, skin-tanning agents, thickeners, vitamins, waxes, fats, phospholipids, saturated fatty acids, mono- or polyunsaturated fatty acids, α-hydroxy acids, polyhydroxy fatty acids, liquefiers, dyes, color-protection agents, pigments, anti-corrosives, polyols, electrolytes, or silicone derivatives.

The compounds (I) or the stereoisomers thereof or the mixture of different stereoisomers thereof or the mixtures of different compounds (I), as described herein (in case of composition A in admixture with the compound (II) or the stereoisomers thereof or the mixture of different stereoisomers thereof) can be used in a wide range of compositions, preferably in aroma compositions, more preferably in fragrance compositions. The olfactory properties and the substance properties (such as solubility in customary solvents and compatibility with further customary constituents of such compositions) of the compounds (I) (and (II) in case of composition A) underline the particular suitability of the combinations for the stated use purposes and compositions.

Suitable compositions are for example perfume compositions, body care compositions (including cosmetic compositions), products for oral and dental hygiene, hygiene articles, cleaning compositions (including dishwashing compositions), textile detergent compositions, compositions for scent dispensers, foods, food supplements, pharmaceutical compositions and crop protection compositions.

Perfume compositions can be selected from fine fragrances, air fresheners in liquid form, gel-like form or a form applied to a solid carrier, aerosol sprays, scented cleaners, perfume candles and oils, such as lamp oils or oils for massage.

Examples for fine fragrances are perfume extracts, Eau de Parfums, Eau de Toilettes, Eau de Colognes, Eau de Solide and Extrait Parfum.

Body care compositions include cosmetic compositions, and can be selected from after-shaves, pre-shave products, splash colognes, solid and liquid soaps, shower gels, shampoos, shaving soaps, shaving foams, bath oils, cosmetic emulsions of the oil-in-water type, of the water-in-oil type and of the water-in-oil-in-water type, such as e.g. skin creams and lotions, face creams and lotions, sunscreen creams and lotions, after-sun creams and lotions, hand creams and lotions, foot creams and lotions, hair removal creams and lotions, after-shave creams and lotions, tanning creams and lotions, hair care products such as e.g. hairsprays, hair gels, setting hair lotions, hair conditioners, hair shampoo, permanent and semi-permanent hair colorants, hair shaping compositions such as cold waves and hair smoothing compositions, hair tonics, hair creams and hair lotions, deodorants and antiperspirants such as e.g. underarm sprays, rollons, deodorant sticks and deodorant creams, products of decorative cosmetics such as e.g. eye-liners, eye-shadows, nail varnishes, make-ups, lipsticks and mascara.

Products for oral and dental hygiene can be selected from toothpaste, dental floss, mouth wash, breath fresheners, dental foam, dental gels and dental strips.

Hygiene articles can be selected from joss sticks, insecticides, repellents, propellants, rust removers, perfumed freshening wipes, armpit pads, baby diapers, sanitary towels, toilet paper, cosmetic wipes, pocket tissues, dishwasher and deodorizer.

Cleaning compositions, such as e.g. cleaners for solid surfaces, can be selected from perfumed acidic, alkaline and neutral cleaners, such as e.g. floor cleaners, window cleaners, dishwashing compositions both for handwashing and machine washing use, bath and sanitary cleaners, scouring milk, solid and liquid toilet cleaners, powder and foam carpet cleaners, waxes and polishes such as furniture polishes, floor waxes, shoe creams, disinfectants, surface disinfectants and sanitary cleaners, brake cleaners, pipe cleaners, limescale removers, grill and oven cleaners, algae and moss removers, mold removers, facade cleaners.

Textile detergent compositions can be selected from liquid detergents, powder detergents, laundry pretreatments such as bleaches, soaking agents and stain removers, fabric softeners, washing soaps, washing tablets.

Food means a raw, cooked, or processed edible substance, ice, beverage or ingredient used or intended for use in whole or in part for human consumption, or chewing gum, gummies, jellies, and confectionaries.

A food supplement is a product intended for ingestion that contains a dietary ingredient intended to add further nutritional value to the diet. A dietary ingredient may be one, or any combination, of the following substances: a vitamin, a mineral, an herb or other botanical, an amino acid, a dietary substance for use by people to supplement the diet by increasing the total dietary intake, a concentrate, metabolite, constituent, or extract. Food supplements may be found in many forms such as tablets, capsules, softgels, gelcaps, liquids, or powders.

Pharmaceutical compositions comprise compositions which are intended for use in the diagnosis, cure, mitigation, treatment, or prevention of disease as well as articles (other than food) intended to affect the structure or any function of the body of man or other animals.

Crop protection compositions comprise compositions which are intended for the managing of plant diseases, weeds and other pests (both vertebrate and invertebrate) that damage agricultural crops and forestry.

The compositions according to the invention can further comprise one or more substances, such as, for example: preservatives, abrasives, anti-acne agents, agents to combat skin aging, antibacterial agents, anti-cellulite agents, antidandruff agents, antiinflammatory agents, irritation-preventing agents, irritation-alleviating agents, antimicrobial agents, antioxidants, astringents, sweat-inhibiting agents, antiseptics, antistatics, binders, buffers, carrier materials, chelating agents, cell stimulants, cleaning agents, care agents, hair removal agents, surface-active substances, deodorizing agents, antiperspirants, emulsifiers, enzymes, essential oils, fibers, film formers, fixatives, foam formers, foam stabilizers, substances for preventing foaming, foam boosters, fungicides, gelling agents, gel-forming agents, hair care agents, hair shaping agents, hair smoothing agents, moisture-donating agents, moisturizing substances, humectant substances, bleaching agents, strengthening agents, stain removal agents, optical brighteners, impregnating agents, soil repellents, friction-reducing agents, lubricants, moisturizing creams, ointments, opacifiers, plasticizers, covering agents, polish, shine agents, polymers, powders, proteins, refatting agents, exfoliating agents, silicones, skin-calming agents, skin-cleansing agents, skin care agents, skin-healing agents, skin lightening agents, skin-protective agents, skin-softening agents, cooling agents, skin-cooling agents, warming agents, skin-warming agents, stabilizers, UV-absorbent agents, UV filters, fabric softeners, suspending agents, skin-tanning agents, thickeners, vitamins, waxes, fats, phospholipids, saturated fatty acids, mono- or polyunsaturated fatty acids, α-hydroxy acids, polyhydroxy fatty acids, liquefiers, dyes, color-protection agents, pigments, anti-corrosives, polyols, electrolytes, or silicone derivatives.

The compounds (I) or the stereoisomers thereof or the mixture of different stereoisomers thereof or the mixtures of different compounds (I), as described herein (and the compound (II) or a stereoisomer thereof or a mixture of different stereoisomers thereof in case of composition A) may be worked into in compositions simply by directly mixing them with the basic composition lacking only this/these compound(s). Alternatively, the compounds (I) or the stereoisomers thereof or the mixture of different stereoisomers thereof or the mixtures of different compounds (I), as described herein (and the compound (II) or a stereoisomer thereof or a mixture of different stereoisomers thereof in case of composition A) may be mixed simultaneously or consecutively with the other components of the composition or with pre-formed mixtures of a part of the other components.

The compounds (I) or the stereoisomers thereof or the mixture of different stereoisomers thereof or the mixtures of different compounds (I), as described herein (in case of composition A in admixture with the compound (II) or the stereoisomers thereof or the mixture of different stereoisomers thereof) may, in an earlier step, be entrapped with an entrapment material, for example, polymers, capsules, microcapsules and nanocapsules, liposomes, film formers, absorbents such as carbon or zeolites, cyclic oligosaccharides and mixtures thereof, or may be chemically bonded to substrates, which are adapted to release the compounds (I) upon application of an external stimulus such as light, enzyme, or the like, and then mixed with the composition.

The compounds (I) or the stereoisomers thereof or the mixture of different stereoisomers thereof or the mixtures of different compounds (I), as described herein (and the compound (II) or a stereoisomer thereof or a mixture of different stereoisomers thereof in case of composition A) and compositions comprising these compounds according to the present invention can also be in microencapsulated form, spray-dried form, in the form of inclusion complexes or in the form of extrusion products. The properties can be further optimized by so-called “coating” with suitable materials with regard to a more targeted release of the scent, for which purpose preferably waxy synthetic substances such as e.g. polyvinyl alcohol are used.

The microencapsulation can take place for example by the so-called coacervation method with the help of capsule materials, e.g. made of polyurethane-like substances or soft gelatin. The spray-dried perfume oils can be produced for example by spray-drying an emulsion or dispersion comprising a compound (I) or a stereoisomer thereof or a mixture of different stereoisomers thereof or a mixtures of different compounds (I), as described herein (and the compound (II) or a stereoisomer thereof or a mixture of different stereoisomers thereof in case of composition A), or a composition of the present invention described herein, wherein carrier substances that can be used are modified starches, proteins, dextrin and vegetable gums. Inclusion complexes can be prepared e.g. by introducing dispersions of fragrance compositions and cyclodextrins or urea derivatives into a suitable solvent, e.g. water. Extrusion products can be produced by melting a compound (I) or a stereoisomer thereof or a mixture of different stereoisomers thereof or a mixtures of different compounds (I), as described herein (in case of composition A in admixture with the compound (II) or a stereoisomer thereof or a mixture of different stereoisomers thereof), or a composition of the present invention described herein with a suitable wax-like substance and by extrusion with subsequent solidification, optionally in a suitable solvent, e.g. isopropanol.

Generally, the total amount of the one or more compounds (I) (and (II) in case of composition A) in the compositions according to the present invention is typically adapted to the particular intended use or the intended application and can, thus, vary over a wide range. As a rule, the customary standard commercial amounts for scents are used.

The compositions according to the invention can comprise the compounds of formula (I) (and (II) in case of composition A) in an overall amount of from 0.001 to 99.9% by weight, preferably from 0.01 to 90% by weight, more preferably from 0.05 to 80%, in particular from 0.1 to 60% by weight, more particularly from 0.1 to 40% by weight, e.g. from 0.1 to 10% by weight or 0.1 to 15% by weight, based on the total weight of the composition.

In one embodiment of the invention, the compositions comprise the compounds of formula (I) (and (II) in case of composition A) in an overall amount of from 0.001 to 5 weight %, preferably from 0.01 to 2 weight % based on the total weight of the composition.

Some of the compounds (I) are novel. The invention relates therefore also to a compound of the formula (I)

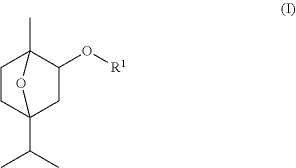

wherein
R¹ is C₁-C₃-alkyl, formyl or ethylcarbonyl;
or a stereoisomer thereof or a mixture of different stereoisomers thereof or a mixture of different compounds (I).

Preferably, R¹ is C₁-C₃-alkyl or ethylcarbonyl, more preferably methyl, ethyl or ethylcarbonyl; specifically ethyl.

As already described in context with compositions A and B, in the compound of the formula (I) the group —OR¹ is preferably bound in exo-position.

The compounds of formula (I) and the mixtures of compounds (I) and (II) not only have a pleasant aroma, especially fragrance; they are also readily available. In particular they can be readily produced with a reduced CO₂ footprint, e.g. by using renewable sources and/or by making use of industrial by-products.

The following examples serve as further illustration of the invention.

### EXAMPLES

### 1. Preparation Examples

### 1.1 Mixture of Carvenone [Compound of Formula (II)] and 2-hydroxy-1,4-cineole (ComPound of Formula (I) Wherein R¹ is H)

A mixture containing 87% by weight of carvenone and 12% by weight of 2-hydroxy-1,4-cineole was obtained as a side product in the synthesis of cinmethylin starting from terpinen-4-ol via epoxidation thereof and acidic rearrangement of the epoxide.

### 1.2 Preparation of a Mixture of Carvenone and (4-isopropyl-1-methyl-7-oxabicyclo[2.2.1]heptan-2-yl) acetate (acetate of 2-hydroxy-1,4-cineole)

20 g (1.00 equivalent) of the mixture of example 1.1 and 0.08 equivalents of N,N-dimethylpyridin-4-amine (DMAP) were placed in 100 ml round bottom flask and heated to 60° C. To this mixture, 1.25 equivalents of acetic anhydride were added within 5 min, and the mixture was stirred at 60° C. for 5 h. Another 0.5 equivalents of acetic anhydride were added, and the mixture was stirred at 60° C. for 20 h. After cooling to room temperature, the reaction mixture was diluted with 25 ml of ethyl acetate and quenched with aqueous saturated NaHCO₃ solution. The phases were separated, the organic phase was washed with aqueous saturated NaHCO₃ and NaCl solution, dried over Na₂SO₄ and the solvent was removed under reduced pressure. Two batches of the crude product of this reaction were purified by distillation to yield fractions with a weight ratio of carvenone to (4-isopropyl-1-methyl-7-oxabicyclo[2.2.1]heptan-2-yl) acetate ranging from 94:5 to 84:15 as colorless liquids in a purity of at least 99.0 GC-a % (GC-a %=percent of the GC area).

### 1.3 Preparation of a Mixture of Carvenone and 2-ethoxy-4-isopropyl-1-methyl-7-oxabicyclo[2.2.1]heptane (=ethyl ether of 2-hydroxy-1,4-cineole)

Carvenone and 2-ethoxy-4-isopropyl-1-methyl-7-oxabicyclo[2.2.1]heptane (=ethyl ether of 2-hydroxy-1,4-cineole; obtained according to example 1.5) were mixed in the desired ratios.

### 1.4 Preparation of (4-isopropyl-1-methyl-7-oxabicyclo[2.2.1]heptan-2-yl) acetate (acetate of 2-hydroxy-1,4-cineole)

50 g (1.00 equivalent) of 2-hydroxy-1,4-cineole and 0.08 equivalents of N,N-dimethylpyridin-4-amine (DMAP) were added to 200 ml of tetrahydrofuran placed in a 1000 ml round bottom flask and heated to 60° C. To this mixture, 1.25 equivalents of acetic anhydride were added within 5 min, and the mixture was stirred at 60° C. for 5 h. After cooling to room temperature, the reaction mixture was diluted with 200 ml of ethyl acetate and quenched with 100 ml aqueous saturated NaHCO₃ solution. The phases were separated, the organic phase was washed with aqueous saturated Na—HCO₃ and NaCl solution, dried over Na₂SO₄ and the solvent was removed under reduced pressure. The crude product was purified by distillation to yield the title compound as a colorless liquid in a purity of >99.9 GC-a %.

¹H NMR (500 MHz, CDCl₃) δ 4.88 (dd, J=7.3, 2.6 Hz, 1H), 2.18 (dd, J=13.2, 7.3 Hz, 1H), 2.13-2.08 (m, 1H), 2.07 (s, 3H), 1.65-1.43 (m, 5H), 1.39 (s, 3H), 1.02-0.92 (m, 6H).

¹³C NMR (126 MHz, CDCl₃) δ 170.8, 88.8, 84.4, 78.4, 77.4, 77.2, 76.9, 43.3, 33.4, 32.5, 31.2, 21.2, 18.2, 17.9, 16.4.

MS (PCI, C₁₂H₂₀O₃) calc. 213.2 [M+H]⁺, found 213.2 [M+H]⁺

### 1.5 Preparation of 2-ethoxy-4-isopropyl-1-methyl-7-oxabicyclo[2.2.1]heptane (=ethyl ether of 2-hydroxy-1,4-cineole)

In a 500 ml round bottom flask, 1.5 equivalents of sodium hydride (60% dispersion in mineral oil) were added to 50 ml of tetrahydrofuran. 20 g (1.00 equivalent) of 2-hydroxy-1,4-cineole in 30 ml of tetrahydrofuran were added to this mixture within 15 minutes. The resulting mixture was stirred for an additional 30 minutes at room temperature. 1.5 equivalents of iodoethane were added within 20 minutes. The mixture was heated to 40° C. and stirred for 23 hours. The mixture was cooled to room temperature and quenched by addition of 50 ml water. The mixture was extracted twice with 60 ml of 2-methoxy-2-methylpropane. The combined organic layers were washed with aqueous saturated NaHCO₃ and NaCl solution, dried over Na₂SO₄ and the solvent was removed under reduced pressure. The crude product was purified by distillation and column chromatography over SiO₂ (cyclohexane/ethyl acetate 95:5 to 90:10) to yield the title compound as a colorless liquid in a purity of >99.9 GC-a %.

¹H NMR (500 MHz, CDCl₃) δ 3.53-3.43 (m, 2H), 3.35 (dq, J=9.3, 7.0 Hz, 1H), 2.09 (h, J=6.9 Hz, 1H), 1.96 (dd, J=12.5, 6.8 Hz, 1H), 1.55 (ddd, J=8.7, 6.6, 3.0 Hz, 2H), 1.50-1.40 (m, 6H), 1.18 (t, J=7.0 Hz, 3H), 0.97 (dd, J=6.9, 2.5 Hz, 6H).

¹³C NMR (126 M Hz, CSCl₃) d 88.5, 85.4, 83.6, 64.1, 42.7, 33.9, 32.7, 31.7, 26.9, 18.3, 18.0, 16.5, 15.4.

MS (PCl, C₁₂H₂₂O₂) calc. 199.2 [M+H]⁺, found 199.2 [M+H]⁺

### 2. Olfactory Tests

In order to test the quality and intensity of the odor of the mixtures of compounds (I) and (II) or compounds (I) only, scent strip tests were performed.

For this purpose, strips of absorbent paper were dipped into a solution containing 1 to 10% by weight solution of the mixture of compounds (I) and (II) or of compound (I) to be tested in tri ethyl citrate. After evaporation of the solvent (about 30 sec.) the scent impression was olfactively evaluated by a trained perfumer.

The results of the scent test are summarized in table 1.

TABLE 1
Results of the scent tests.

| Example | | Odor |
|---|---|---|
| no. | Compound/mixture | Description |
| 1.1 | 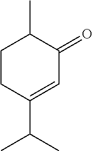 | caraway, terpenic, spicy, woody |
| | 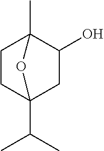 | |
| | Mixture of carvenone and 2-hydroxy-1,4- | |
| | cineole | |
| | in a weight ratio of 87:12 | |
| 1.2 | 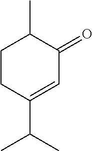 | powdery, caraway, woody |
| | 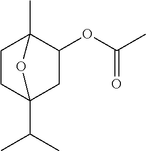 | |
| | Mixture of carvenone and (4-isopropyl-1- | |
| | methyl-7-oxabicyclo[2.2.1]heptan-2-yl) | |
| | acetate in a weight ratio of 87:12 | |
| 1.3 | 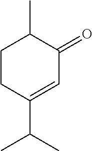 | powdery, caraway with citrus and fruity aspects |
| | 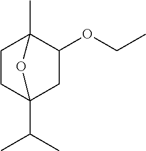 | |
| | Mixture of carvenone and | |
| | 2-ethoxy-4-isopropyl- | |
| | 1-methyl-7-oxabicyclo[2.2.1]heptane | |
| | in a weight ratio of 87:12 | |
| 1.4 | 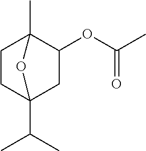 | ambrinol, animalic, civet, soft |
| | (4-isopropyl-1-methyl-7- | |
| | oxabicyclo[2.2.1]heptan-2-yl) acetate | |
| 1.5 | 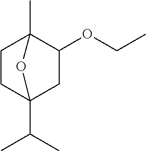 | herbal, incense |
| | 2-ethoxy-4-isopropyl-1-methyl-7- | |
| | oxabicyclo[2.2.1]heptane | |
| 1.6* | 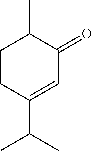 | With increasing amount of 2- hydroxy-1,4- cineole the scent becomes milder. Between 25:75 and 1:99, the impression is less spicy and more fruity. |
| | 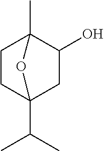 | |
| | Mixtures of carvenone and 2-hydroxy-1,4- | |
| | cineole in various weight ratios | |
| | between 99:1 to 1:99 | |
| 1.7* | 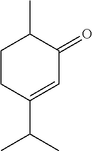 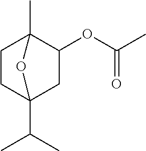 | With increasing amount of (4- isopropyl-1- methyl-7- oxabicyclo [2.2.1]- heptan-2-yl) acetate the scent becomes milder. Between 25:75 and 1:99, the impression is less spicy and more fruity. |
| | Mixtures of carvenone and (4-isopropyl-1- | |
| | methyl-7-oxabicyclo[2.2.1] | |
| | heptan-2-yl) acetate in various | |
| | weight ratios between 99:1 to 1:99 | |
| 1.8** | 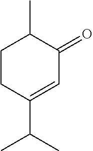 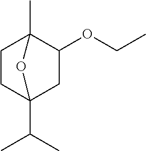 | With increasing amount of 2-ethoxy- 4-isopropyl- 1-methyl-7- oxabicyclo [2.2.1]heptane the scent becomes milder. Between 50:50 and 1:99, the impression is less spicy and more citrus and fruity. |
| | Mixture of carvenone and | |
| | 2-ethoxy-4-isopropyl- | |
| | 1-methyl-7-oxabicyclo[2.2.1]heptane | |
| | in various weight ratios | |
| | between 99:1 to 1:99 | |

*Obtained by mixing the single compounds or by supplementing the mixtures obtained in preparation examples 1.1 or 1.2 with that compound I or II required for obtaining the desired mixing ratio
**Obtained by mixing the single compounds

### Sales Products and Advantageous Fragrance Compositions

“Solution A” is the non-diluted product of example 1.1.
“Solution B” is the non-diluted product of example 1.2.
“Solution C” is the non-diluted product of example 1.3.
“Solution D” is the non-diluted product of example 1.4.
“Solution E” is the non-diluted product of example 1.5.
“Solution F” is the non-diluted product of example 1.6.
“Solution G” is the non-diluted product of example 1.7.
“Solution H” is the non-diluted product of example 1.8.

### Advantageous Fragrance Compositions:

Solution A as described above was formulated in the compositions according to table 2. The amounts given in table 2 are weight units in grams.

TABLE 2
Fragrance compositions 1A and 1B

| | 1A | 1B |
|---|---|---|
| Lactone C10 gamma (5-hexyloxolan-2-one) | 2 | 2 |
| Bourgeonal (3-(4-tert-butylphenyl)propanal) | 2 | 2 |
| Citronellol | 3 | 3 |
| Aldehyde C-14 (5-heptyloxolan-2-one) | 3 | 3 |
| Allyl heptylate | 4 | 4 |
| Amber core (1-(2-tert-butylcyclohexyl)oxybutan-2-ol) | 4 | 4 |
| Ethyl-2-methyl butyrate | 4 | 4 |
| Geranyl acetate | 5 | 5 |
| Helional ® (3-(1,3-benzodioxol-5-yl)-2-methylpropanal) | 10 | 10 |
| Manzanate (ethyl 2-methylpentanoate) | 10 | 10 |
| Amberwood ® (ethoxymethoxycyclododecane) | 10 | 10 |
| Hexyl acetate | 11 | 11 |
| Benzyl salicylate | 12 | 12 |
| Magnolan ® (2,4-dimethyl-4,4a,5,9b- | 15 | 15 |
| tetrahydroindeno[1,2-d][1,3]dioxine) | | |
| Verdox ® (2-tert-butylcyclohexyl) acetate) | 25 | 25 |
| Bergamot oil bergaptene free | 25 | 25 |
| Linalol | 30 | 30 |
| Dipropylene glycol | 45 | 45 |
| Iso E Super ® (Tetramethyl acetyloctahydronaphthalenes) | 110 | 110 |
| Pyranol (4-methyl-2-(2-methylpropyl)oxan-4-ol) | 170 | 170 |
| Hedione ® (methyl 3-oxo-2-pentylcyclopentaneacetate) | 200 | 200 |
| Galaxolide ® 50% IPM (1,3,4,6,7,8-hexahydro- | 300 | 300 |
| 4,6,6,7,8,8-hexamethylcyclopenta(g)-2-benzopyran | | |
| 50% in isopropyl myristate) | | |
| Solution A | 25 | 50 |
| | 1025 | 1050 |

Solution A as described above was formulated in the compositions according to table 3. The amounts given in table 3 are weight units in grams.

TABLE 3
Fragrance compositions 2A and 2B

| | 2A | 2B |
|---|---|---|
| Raspberry ketone (4-(4-hydroxyphenyl)butan-2-one) | 4 | 4 |
| Vanitrope ® (2-ethoxy-5-prop-1-enylphenol) | 6 | 6 |
| Cyclamen aldehyde (at least 90% | 10 | 10 |
| 2-methyl-3-(p-isopropylphenyl)- | | |
| propionaldehyde; secondary component: 5% | | |
| 3-(p-cumenyl)-2-methylpropionic acid) | | |
| Bicyclononalactone (3,4,4a,5,6,7,8,8a- | 10 | 10 |
| octahydrochromen-2-one) | | |
| Aldehyde C-14 (5-heptyloxolan-2-one) | 14 | 14 |
| Ethylvanillin (3-ethoxy-4-hydroxybenzaldehyde) | 16 | 16 |
| Heliotropine (1,3-benzodioxole-5-carbaldehyde) | 20 | 20 |
| Iso E Super ® (tetramethyl acetyloctahydronaphthalenes) | 20 | 20 |
| Sandela ® (3-[5,5,6-trimethylbicyclo[2.2.1]hept- | 30 | 30 |
| 2-yl]cyclohexan-1-ol) | | |
| Vanillin isobutyrate ((4-formyl-2-methoxyphenyl) 2- | 40 | 40 |
| methylpropanoate) | | |
| Aldehyde C-18 (5-pentyloxolan-2-one) | 50 | 50 |
| Benzyl salicylate | 60 | 60 |
| Hexyl cinnamic aldehyde (2-(phenylmethylidene)octanal) | 70 | 70 |
| Hedione ® (methyl 3-oxo-2-pentylcyclopentaneacetate) | 130 | 130 |
| Pyranol (4-methyl-2-(2-methylpropyl)oxan-4-ol) | 150 | 150 |
| Ethylene brassylate (1,4- | 170 | 170 |
| dioxacycloheptadecane-5,17-dione) | | |
| Galaxolide ® 50% IPM (1,3,4,6,7,8-hexahydro- | 200 | 200 |
| 4,6,6,7,8,8-hexamethylcyclopenta(g)-2-benzopyran | | |
| 50% in isopropyl myristate) | | |
| Solution A | 10 | 20 |
| | 1010 | 1020 |

Fragrance composition 3 corresponds to fragrance composition 1A, where Solution A is replaced by the same amount of Solution B. Fragrance composition 4 corresponds to fragrance composition 1B, where Solution A is replaced by the same amount of Solution B. Fragrance composition 5 corresponds to fragrance composition 2A, where Solution A is replaced by the same amount of Solution B. Fragrance composition 6 corresponds to fragrance composition 2B, where Solution A is replaced by the same amount of Solution B.

Fragrance composition 7 corresponds to fragrance composition 1A, where Solution A is replaced by the same amount of Solution C. Fragrance composition 8 corresponds to fragrance composition 1B, where Solution A is replaced by the same amount of Solution C. Fragrance composition 9 corresponds to fragrance composition 2A, where Solution A is replaced by the same amount of Solution C. Fragrance composition 10 corresponds to fragrance composition 2B, where Solution A is replaced by the same amount of Solution C.

Fragrance composition 11 corresponds to fragrance composition 1A, where Solution A is replaced by the same amount of Solution D. Fragrance composition 12 corresponds to fragrance composition 1B, where Solution A is replaced by the same amount of Solution D. Fragrance composition 13 corresponds to fragrance composition 2A, where Solution A is replaced by the same amount of Solution D. Fragrance composition 14 corresponds to fragrance composition 2B, where Solution A is replaced by the same amount of Solution D.

Fragrance composition 15 corresponds to fragrance composition 1A, where Solution A is replaced by the same amount of Solution E. Fragrance composition 16 corresponds to fragrance composition 1B, where Solution A is replaced by the same amount of Solution E. Fragrance composition 17 corresponds to fragrance composition 2A, where Solution A is replaced by the same amount of Solution E. Fragrance composition 18 corresponds to fragrance composition 2B, where Solution A is replaced by the same amount of Solution E.

Fragrance composition 19 corresponds to fragrance composition 1A, where Solution A is replaced by the same amount of Solution F. Fragrance composition 20 corresponds to fragrance composition 1B, where Solution A is replaced by the same amount of Solution F. Fragrance composition 21 corresponds to fragrance composition 2A, where Solution A is replaced by the same amount of Solution F. Fragrance composition 22 corresponds to fragrance composition 2B, where Solution A is replaced by the same amount of Solution F.

Fragrance composition 23 corresponds to fragrance composition 1A, where Solution A is replaced by the same amount of Solution G. Fragrance composition 24 corresponds to fragrance composition 1B, where Solution A is replaced by the same amount of Solution G. Fragrance composition 25 corresponds to fragrance composition 2A, where Solution A is replaced by the same amount of Solution G. Fragrance composition 26 corresponds to fragrance composition 2B, where Solution A is replaced by the same amount of Solution G.

Fragrance composition 27 corresponds to fragrance composition 1A, where Solution A is replaced by the same amount of Solution H. Fragrance composition 28 corresponds to fragrance composition 1B, where Solution A is replaced by the same amount of Solution H. Fragrance composition 29 corresponds to fragrance composition 2A, where Solution A is replaced by the same amount of Solution H. Fragrance composition 30 corresponds to fragrance composition 2B, where Solution A is replaced by the same amount of Solution H.

The compositions according to tables 2 and table 3, namely 1A, 1B, 2A and 2B as well as fragrance compositions 3, 4, 5, 6, 7, 8, 9, 10, 11, 12, 13, 14, 15, 16, 17, 18, 19, 20, 21, 22, 23, 24, 25, 26, 27, 29 and 30 could be included in various compositions selected from the group consisting of deo pump spray, clean hair-conditioner, face wash gel, foam bath concentrate, hair gel, self-foaming bodywash, sprayable sun care emulsion, sprayable sun protection emulsion, emollient facial gel, 2-phases oil foam bath, shampoos, shower bath, hydro-alcoholic AP/deo pump spray, aerosol, aqueous/alcoholic AP/deo roll-on, styling gel type “Out of Bed”, shaving foam, sensitive skin baby shampoo, body wash for sensitive skin, gloss enhancing shampoo for sensitive scalp, deo stick, baby wipe, after shave balm, face gel, face day care cream, face cleanser, body lotion, sun care SPF50+, sprayable lotion, hand dish cleaner—regular, hand dish cleaner—concentrate, sanitary cleaner—concentrate, all-purpose cleaner, anti-bacterial fabric softener, detergent composition, powder detergent composition and liquid detergent composition.

A person skilled in art may be well versed with the various general formulations for the above-mentioned products.

Compositions 1A, 1B, 2A, 2B, 3, 4, 5, 6, 7, 8, 9, 10, 11, 12, 13, 14, 15, 16, 17, 18, 19, 20, 21, 22, 23, 24, 25, 26, 27, 29 and 30 can for example be formulated in specific formulations as disclosed in IP.com Number: IPCOM000258614D entitled New Aroma Chemicals pages 6 to 46, Table 1 to Table D13, wherein the “Fragrance Composition 1A” is replaced by identical amounts of compositions 1A, 1B, 2A, 2B, 3, 4, 5, 6, 7, 8, 9, 10, 11, 12, 13, 14, 15, 16, 17, 18, 19, 20, 21, 22, 23, 24, 25, 26, 27, 29 or 30.

1.-22. (canceled)

23. The use of a composition comprising
(a) a compound of the formula (I) 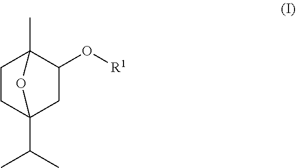
wherein
R¹ is hydrogen, C₁-C₄-alkyl, formyl or C₁-C₃-alkylcarbonyl;
or a stereoisomer thereof or a mixture of different stereoisomers thereof or a mixture of different compounds (I); and
(b) a compound of the formula (II) 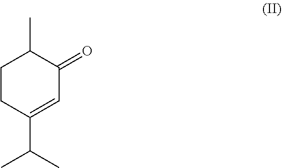
or a stereoisomer thereof or mixture of different stereoisomers thereof;
as an aroma chemical.

24. The use according to claim 23, where in the compound of the formula (I) R¹ is hydrogen.

25. The use according to claim 23, where in the compound of the formula (I) R¹ is formyl or C₁-C₃-alkylcarbonyl.

26. The use according to claim 23, where in the compound of the formula (I) R¹ is C₁-C₄-alkyl.

27. The use according to claim 23, where in the compound of the formula (I) the group —OR¹ is bound in exo-position.

28. The use according to claim 23, where the compound of the formula (I) and the compound of the formula (II) are used in a molar ratio of from 100:1 to 1:100.

29. The use according to claim 23, as a fragrance.

30. The use of a composition as defined in claim 23 for modifying and/or enhancing the aroma of a composition.

31. A composition comprising:
(a) a compound of the formula (I) as defined in claim 23 or a stereoisomer thereof or a mixture of different stereoisomers thereof or a mixture of different compounds (I), and
(b) a compound of the formula (II) as defined in claim 23 or a stereoisomer thereof or mixture of different stereoisomers thereof.

32. The composition according to claim 31, where in case that in the compound of the formula (I) R¹ is hydrogen, the compound of the formula (I) and the compound of the formula (II) are present in a molar ratio of from 3:1 to 1:100.

33. The composition according to claim 31, where in case that in the compound of the formula (I) R¹ is not hydrogen, the compound of the formula (I) and the compound of the formula (II) are present in a molar ratio of from 100:1 to 1:100.

34. The composition according to claim 31, further comprising at least one further component selected from the group consisting of aroma chemicals different from compounds (I) and (II), non-aroma chemical carriers, anti-oxidants and deodorant-active agents.

35. A composition comprising a compound of the formula (I) as defined in claim 23, or a stereoisomer thereof or a mixture of different stereoisomers thereof or a mixture of different compounds (I), and at least one further component selected from the group consisting of aroma chemicals different from compound (I), non-aroma chemical carriers, anti-oxidants and deodorant-active agents;
with the proviso that R¹ is not hydrogen if the composition comprises menthol, 4-hydroxycarvomenthone, 3,7-dimethyl-2,6-octadiene, cryptone, p-menthane-1,2-diol, toluene, dichloromethane, tetrahydrofuran, dimethylformamide, hexane or chloroform;
with the proviso that R¹ is not acetyl (methylcarbonyl; —C(O)CH₃) if the composition comprises dichloromethane or hexane; and
with the proviso that in case that R¹ is hydrogen and the aroma chemicals different from compound (I) is a compound of the formula (II) as defined in claim 23, the compound of the formula (I) and the compound of the formula (II) are present in a molar ratio of from 3:1 to 1:>1.

36. The composition according to claim 35, where in the compound of the formula (I) R¹ is C₁-C₄-alkyl, formyl or C₁-C₃-alkylcarbonyl.

37. The composition according to claim 34, where in case that the non-aroma chemical carriers comprise one or more solvents, these are selected from the group consisting of ethanol, isopropanol, dipropylene glycol (DPG), propylene glycol, 1,2-butylene glycol, glycerol, diethylene glycol monoethyl ether, diethyl phthalate (DEP), isopropyl myristate (IPM), triethyl citrate (TEC), benzyl benzoate and mixtures thereof.

38. The composition according to claim 34, where the aroma chemicals different from compounds (I) and (II) are selected from the group consisting of geranyl acetate, alpha-hexylcinnamaldehyde, 2-phenoxyethyl isobutyrate, dihydromyrcenol, methyl dihydrojasmonate, 4,6,6,7,8,8-hexamethyl-1,3,4,6,7,8-hexahydrocyclopenta[g]benzopyran, tetrahydrolinalool, ethyllinalool, benzyl salicylate, 2-methyl-3-(4-tert-butylphenyl)propanal, cinnamyl alcohol, 4,7-methano-3a,4,5,6,7,7a-hexahydro-5-indenyl acetate, 4,7-methano-3a,4,5,6,7,7a-hexahydro-6-indenyl acetate, citronellol, citronellyl acetate, tetrahydrogeraniol, vanillin, linalyl acetate, styrolyl acetate, octahydro-2,3,8,8-tetramethyl-2-acetonaphthone, 2-acetyl-1,2,3,4,6,7,8-octahydro-2,3,8,8-tetramethylnaphthalene, hexyl salicylate, 4-tert-butylcyclohexyl acetate, 2-tert-butylcyclohexyl acetate, alpha-ionone, n-alpha-methylionone, alpha-isomethylionone, coumarin, terpinyl acetate, 2-phenylethyl alcohol, 4-(4-hydroxy-4-methylpentyl)-3-cyclohexenecarboxaldehyde, alpha-amylcinnamaldehyde, ethylene brassylate, (E)-3-methylcyclopentadec-5-enone, (Z)-3-methylcyclopentadec-5-enone, 15-pentadec-11-enolide, 15-pentadec-12-enolide, 15-cyclopentadecanolide, 1-(5,6,7,8-tetrahydro-3,5,5,6,8,8-hexamethyl-2-naphthalenyl)ethanone, 2-isobutyl-4-methyltetrahydro-2H-pyran-4-ol, 2-ethyl-4-(2,2,3-trimethyl-3-cyclopenten-1-yl)-2-buten-1-ol, cis-3-hexenyl acetate, trans-3-hexenyl acetate, trans-2/cis-6-nonadienol, 2,4-dimethyl-3-cyclohexenecarboxaldehyde, 2,4,4,7-tetramethyloct-6-en-3-one, 2,6-dimethyl-5-hepten-1-al, borneol, 3-(3-isopropylphenyl)butanal, 2-methyl-3-(3,4-methylenedioxyphenyl)propanal, 3-(4-ethylphenyl)-2,2-dimethylpropanal, 7-methyl-2H-1,5-benzodioxepin-3(4H)-one, 3,3,5-trimethylcyclohexyl acetate, 2,5,5-trimethyl-1,2,3,4,4a,5,6,7-octahydronaphthalen-2-ol, 3-(4-tert-butylphenyl)-propanal, ethyl 2-methylpentanoate, ethoxymethoxycyclododecane, 2,4-dimethyl-4,4a,5,9b-tetrahydroindeno[1,2-d][1,3]dioxine, (2-tert-butylcyclohexyl) acetate, 3-[5,5,6-trimethylbicyclo[2.2.1]hept-2-yl]cyclohexan-1-ol,
menthone, isomenthone, carvone, camphor, beta-ionone, beta-n-methylionone, beta-isomethylionone, alpha-irone, alpha-damascone, beta-damascone, beta-damascenone, delta-damascone, acetylated cedar wood oil, and mixtures thereof.

39. The composition according to claim 31, which is selected from the group consisting of perfume compositions, body care compositions, products for oral or dental hygiene, hygiene articles, cleaning compositions, textile detergent compositions, compositions for scent dispensers, foods, food supplements, pharmaceutical compositions and crop protection compositions.

40. The use of a compound of the formula (I) as defined in claim 23 or of a stereoisomer thereof or of a mixture of different stereoisomers thereof or of a mixture of different compounds (I), as an aroma chemical, where the use does not encompass the use of the compound of the formula (I) where R¹ is hydrogen as an aroma chemical in essential oil obtained from the rhizomes of Ferula jaeschkeana.

41. The use of a compound of the formula (I) as defined in claim 23 or of a stereoisomer thereof or of a mixture of different stereoisomers thereof or of a mixture of different compounds (I) for modifying and/or enhancing the aroma of a composition;
where the use does not encompass the use of the compound of the formula (I) where R¹ is hydrogen for modifying and/or enhancing the aroma of menthol or for modifying and/or enhancing the aroma of essential oil obtained from the rhizomes of Ferula jaeschkeana.

42. The use according to claim 40, where in the compound of the formula (I) R¹ is C₁-C₄-alkyl, formyl or C₁-C₃-alkylcarbonyl.

43. The use according to claim 40, where in the compound of the formula (I) R¹ is hydrogen, where the use does not encompass the use of the compound of the formula (I) where R¹ is hydrogen for modifying and/or enhancing the aroma of menthol or as an aroma chemical in essential oil obtained from the rhizomes of Ferula jaeschkeana or for modifying and/or enhancing the aroma of essential oil obtained from the rhizomes of Ferula jaeschkeana.

44. A compound of the formula (I) 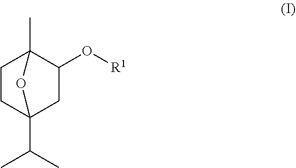
wherein
R¹ is C₁-C₃-alkyl, formyl or ethylcarbonyl;
or a stereoisomer thereof or a mixture of different stereoisomers thereof or a mixture of different compounds (I).
